(19) United States
(12) Patent Application Publication
(10) Pub. No.: US 2006/0167046 A1
(43) Pub. Date: Jul. 27, 2006

(54) Histamine h3 receptor antagonists, preparation and therapeutic uses

(75) Inventors: Lisa Selsam Beavers, Franklin, IN (US); Don Richard Finley, Greenwood, IN (US); Robert Alan Gadski, Indianapolis, IN (US); Philip Arthur Hipskind, New Palestine, IN (US); Cynthia Darshini Jesudason, Indianapolis, IN (US); Richard Todd Pickard, Noblesville, IN (US); Freddie Craig Stevens, Indianapolis, IN (US)

Correspondence Address: ELI LILLY & COMPANY, PATENT DIVISION, P.O. BOX 6288, INDIANAPOLIS, IN, 46206-6288, US

(73) Assignee: Eli Lilly and Company Patent Division, Indianapolis, IN (US)

(21) Appl. No.: 10/525,315

(22) PCT Filed: Sep. 12, 2003
(86) PCT No.: PCT/US03/25860

§ 371 Date: Feb. 23, 2005

Related U.S. Application Data
(60) Provisional application No. 60/411,625, filed on Sep. 18, 2002.

(51) Int. Cl. A61K 31/4709; A61K 31/454; A61K 31/405; C07D 401/02; C07D 403/02

(52) U.S. Cl. 514/312; 514/323; 546/153; 546/201; 514/414; 548/465

(57) ABSTRACT

The present invention discloses novel compounds of Formula (I) or pharmaceutically acceptable salts thereof which have histamine-II3 receptor antagonist activity as well as methods for preparing such compounds. In another embodiment, the invention discloses pharmaceutical compositions comprising compounds of Formula (I), as well as methods or using them to treat obesity and other histamine II3 receptor-related diseases.

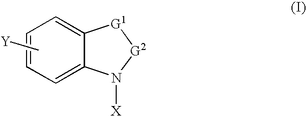

The present invention relates to histamine H3 receptor antagonists, and as such are useful in the treatment of disorders responsive to the inactivation of histamine H3 receptors, such as obesity, cognitive disorders, attention deficit disorders and the like.

The histamine H3 receptor (H3R) is a presynaptic autoreceptor and hetero-receptor found in the peripheral and central nervous system and regulates the release of histamine and other neurotransmitters, such as serotonin and acetylcholine. The histamine H3 receptor is relatively neuron specific and inhibits the release of a number of monamines, including histamine. Selective antagonism of the histamine H3 receptor raises brain histamine levels and inhibits such activities as food consumption while minimizing non-specific peripheral consequences. Antagonists of the histamine H3 receptor increase synthesis and release of cerebral histamine and other monoamines. By this mechanism, they induce a prolonged wakefulness, improved cognitive function, reduction in food intake and normalization of vestibular reflexes. Accordingly, the histamine H3 receptor is an important target for new therapeutics in Alzheimer disease, mood and attention adjustments, cognitive deficiencies, obesity, dizziness, schizophrenia, epilepsy, sleeping disorders, narcolepsy and motion sickness.

The majority of histamine H3 receptor antagonists to date resemble histamine in possessing an imidazole ring generally substituted in the 4(5) position (Ganellin et al., Ars Pharmaceutica, 1995, 36:3, 455-468). A variety of patents and patent applications directed to antagonists and agonists having such structures include EP 197840, EP 494010, WO 97/29092, WO 96/38141, and WO96/38142. These imidazole-containing compounds have the disadvantage of poor blood-brain barrier penetration, interaction with cytochrome P450 proteins, and hepatic and ocular toxicities.

Non-imidazole neuroactive compounds such as beta histamines (Arrang, Eur. J. Pharm. 1985, 111:72-84) demonstrated some histamine H3 receptor activity but with poor potency. EP 978512 published Mar. 1, 2000 discloses non-imidazole aryloxy alkylamines discloses histamine H3 receptor antagonists but does not disclose the affinity, if any, of these antagonists for recently identified histamine receptor GPRv53, described below. EP 0982300A2 (pub. Mar. 1, 2000) discloses non-imidazole alkyamines as histamine HS receptor ligands which have a phenoxy core structure. The subject invention is unique in the presence of a saturated, fused heterocyclic ring appended to the central benzene core. Furthermore the compounds of this invention are selective for the H3 receptor (vs. other histamine receptors).

Histamine mediates its activity via four receptor subtypes, H1R, H2R, H3R and a newly identified receptor designated GPRv53 [(Oda T., et al., J. Biol. Chem. 275 (47): 36781-6 (2000)]. Although relatively selective ligands have been developed for H1R, H2R and H3R, few specific ligands have been developed that can distinguish H3R from GPRv53. GPRv53 is a widely distributed receptor found at high levels in human leukocytes. Activation or inhibition of this receptor could result in undesirable side effects when targeting antagonism of the H3R receptor. Furthermore, the identification of this new receptor has fundamentally changed histamine biology and must be considered in the development of histamine H3 receptor antagonists.

Because of the unresolved deficiencies of the compounds described above, there is a continuing need for improved methods and compositions to treat disorders associated with histamine H3 receptors.

The present invention provides compounds that are useful as histamine H3 receptor antagonists. In another aspect, the present invention provides compounds that are useful as selective antagonists of the histamine H3 receptor relative to other histamine receptors. In yet another aspect, the present invention provides pharmaceutical compositions comprising antagonists of the histamine H3 receptor.

In yet another aspect, the present invention provides compounds, pharmaceutical compositions, and methods useful in the treatment of obesity, cognitive disorders, attention deficit disorders and other disorders associated with histamine H3 receptor.

The present invention is a compound structurally represented by Formula I A compound structurally represented by Formula I 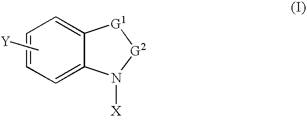
or pharmaceutically acceptable salts thereof wherein:
G¹ is —CH₂—, or —CH₂—CH₂—,
G² is —CH₂—, or —C(O)—,
or G¹ and G² taken together combine to form —CH═CH— or —CH₂—CH═CH—,
Y is
—OCH₂CH₂N-piperidinyl,
—OCH₂CH₂CH₂N-piperidinyl,
—OCH₂CH₂N-pyrrolidinyl,
—OCH₂CH₂CH₂N-pyrrolidinyl,
   X is H, —COR³, —CH₂R⁴, —SO₂R⁵,
   R³ is
—(C₁-C₈) alkyl, optionally substituted with 1 to 3 halogens,
—(C₃-C₈) cycloalkyl, optionally substituted with 1 to 3 halogens,
—O(C₁-C₈) alkyl, optionally substituted with 1 to 3 halogens, 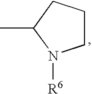
   wherein R⁶ is —(C₁-C₆) alkyl, or —COO—(C₁-C₆) alkyl,
-Furanyl,
-Thienyl,
—NH-phenyl,
—NH—(C₁-C₄)alkyl-phenyl,
—NH—(C₁-C₈) alkyl, optionally substituted with 1 to 4 halogens,
—NH—(C₃-C₈) cycloalkyl, optionally substituted once or twice with halogens,
—CH₂-Pyridinyl,
—CH₂N(C₁-C₆) alkyl (C₁-C₆) alkyl,
—CH₂N-phenyl,
   R⁴ is
—(C₁-C₈) alkyl, optionally substituted with 1 to 4 halogens,
—(C₃-C₈) cycloalkyl,
—(C₁-C₈) alkyl-NH₂,
—(C₁-C₄) alkyl-phenyl,
—CH₂N-phenyl,
-phenyl-O—(C₁-C₄) alkyl-phenyl,
—(C₁-C₄) alkyl-O—(C₁-C₄) alkyl-phenyl,
—CH₂NCO₂—(C₁-C₆) alkyl,
-Phenyl,
-Thienyl,
-Furanyl,
-Imidazolyl,
-Naphthyl, 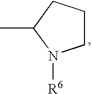
   wherein R⁶ is —(C₁-C₆) alkyl, or —COO—(C₁-C₆) alkyl,
-Biphenyl, and
   R⁵ is
-Phenyl,
—(C₁-C₄) alkyl,
—(C₁-C₄) alkyl-phenyl.

The present invention is a pharmaceutical composition which comprises a compound of Formula I and a pharmaceutically acceptable carrier. Pharmaceutical formulations of Formula I can provide a method of selectively increasing histamine levels in cells by contacting the cells with an antagonist of the histamine H3 receptor, the antagonists being a compound of Formula I. Thus, the methods of this invention encompass a prophylactic and therapeutic administration of a compound of Formula I.

The present invention further provides an antagonist of Formula I which is characterized by selectively binding the histamine receptor H3R as compared to the histamine receptor GPRv53. Thus, a pharmaceutical preparation of Formula I can be useful in the treatment or prevention of obesity, cognitive disorders, attention deficit disorders and the like, which comprises administering to a subject in need of such treatment or prevention an effective amount of a compound of Formula I. In addition, a pharmaceutical preparation of Formula I can be useful in the treatment or prevention of a disorder or disease in which inhibition of the histamine H3 receptor has a beneficial effect or the treatment or prevention of eating disorders which comprises administering to a subject in need of such treatment or prevention an effective amount of a compound of Formula I.

General terms used in the description of compounds, compositions, and methods herein described, bear their usual meanings. Throughout the instant application, the following terms have the indicated meanings:

The term “GPRv53” means a recently identified novel histamine receptor as described in Oda, et al., supra. Alternative names for this receptor are PORT3 or H4R.

The term “H3R” means to the histamine H3 receptor that inhibits the release of a number of monoamines, including histamine.

The term “H1R” means to the histamine H1 receptor subtype.

The term “H2R” means to the histamine H2 receptor subtype.

The term “selective H3R antagonists” is defined as the ability of a compound of the present invention to block forskolin-stimulated cAMP production in response to agonist R (−)α methylhistamine.

In the general formulae of the present document, the general chemical terms have their usual meanings. For example;

“Boc” or “BOC” refer to t-butyl carbamate.

“HOBt” is 1-hydrobenzotriazole.

“PS-Trisamine” is Tris-(2-aminoethyl)amine polystyrene. “PS-Carbodiimide” or “PS-CDI” is N-Cyclohexylcarbodiimide-N′-propyloxymethyl polystyrene. “PS-DIEA” is N,N-(Diisopropyl)aminomethylpolystyrene (1% inorganic antistatic agent). “PS-DMAP” is N-(methylpolystyrene)-4-(methylamino) pyridine.

“Alkylene” are a saturated hydrocarbyldiyl radical of straight or branched configuration made up of from 1 to 4 carbon atoms. Included within the scope of this term are methylene, 1,2-ethane-diyl, 1,1-ethane-diyl, 1,3-propane diyl, 1,2-propane diyl, 1,3 butane-diyl, 1,4-butane diyl, and the like.

“C₃-C₇ cycloalkylene” are a saturated hydrocarbyldiyl radical of cyclic configuration, optionally branched, made up of from 3 to 7 carbon atoms. Included within the scope of this term are cyclopropyl, cyclobutyl, cyclopentyl and cyclohexyl, and the like.

“Alkyl” are one to eight carbon atoms such as methyl, ethyl, propyl, butyl, pentyl, hexyl, heptyl, octyl, and the like, and isomeric forms thereof.

“Aryl” are six to twelve carbon atoms such as phenyl, alpha-naphthyl, beta-naphthyl, m-methylphenyl, p-trifluoromethylphenyl and the like. The aryl groups can also be substituted with one to 3 hydroxy, fluoro, chloro, or bromo groups.

“Cycloalkyl” are three to eight carbon atoms such as cyclopropyl, cyclobutyl, cyclopentyl, cyclohexyl, and the like.

“—CH₂N-pyrrolidinyl” is;

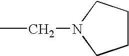

“—CH₂N-piperidinyl” is;

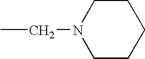

“-phenyl-O—(C₁-C₄) alkyl-phenyl” is;

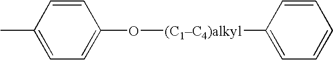

“—(C₁-C₄) alkyl-O—(C₁-C₄) alkyl-phenyl” is;

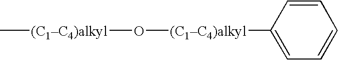

“Halogen” or “halo” means fluoro, chloro, bromo and iodo.

“Composition” means a pharmaceutical composition and is intended to encompass a pharmaceutical product comprising the active ingredient(s), Formula I, and the inert ingredient(s) that make up the carrier. Accordingly, the pharmaceutical compositions of the present invention encompass any composition made by admixing a compound of the present invention and a pharmaceutically acceptable carrier.

The term “unit dosage form” means physically discrete units suitable as unitary dosages for human subjects and other non-human animals, each unit containing a predetermined quantity of active material calculated to produce the desired therapeutic effect, in association with a suitable pharmaceutical carrier.

The terms “treating” and “treat,” as used herein, include their generally accepted meanings, i.e., preventing, prohibiting, restraining, alleviating, ameliorating, slowing, stopping, or reversing the progression or severity of a pathological condition, described herein.

While all of the compounds of the present invention are useful, certain of the compounds are particularly interesting and are preferred. The following listing sets out several groups of preferred compounds. In one embodiment, the present invention provides compounds of Formula I as described in detail above. Other embodiments include the following, wherein the listings set out several groups of preferred compounds. It will be understood that each of the listings may be combined with other listings to create additional groups of preferred embodiments.

1. Wherein G¹ is —CH₂—.

2. Wherein G¹-CH₂—CH₂—.

3. Wherein Y is in the 5 position.

4. Wherein Y is in the 6 position.

5. Wherein X is —COR³.

6. A compound of Formula II 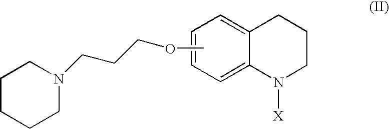
7. A compound of Formula III

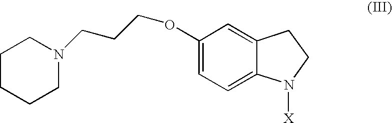

The invention includes tautomers, enantiomers and other stereoisomers of the compounds also. Thus, as one skilled in the art knows, certain aryls may exist in tautomeric forms. Such variations are contemplated to be within the scope of the invention. It will be understood that, as used herein, references to the compounds of Formula I are meant to also include the pharmaceutical salts, its enantiomers and racemic mixtures thereof.

As used herein, the term “stereoisomer” refers to a compound made up of the same atoms bonded by the same bonds but having different three-dimensional structures which are not interchangeable. The three-dimensional structures are called configurations. As used herein, the term “enantiomer” refers to two stereoisomers whose molecules are nonsuperimposable mirror images of one another. The term “chiral center” refers to a carbon atom to which four different groups are attached. As used herein, the term “diastereomers” refers to stereoisomers which are not enantiomers. In addition, two diastereomers which have a different configuration at only one chiral center are referred to herein as “epimers.” The terms “racemate,” “racemic mixture” or “racemic modification” refer to a mixture of equal parts of enantiomers.

The term “enantiomeric enrichment” as used herein refers to the increase in the amount of one enantiomer as compared to the other. A convenient method of expressing the enantiomeric enrichment achieved is the concept of enantiomeric excess, or “ee,” which is found using the following equation:

$$ee = \frac{{E}^{1} - {E}^{2}}{{E}^{1} + {E}^{2}} \times 100$$

wherein E¹ is the amount of the first enantiomer and E² is the amount of the second enantiomer. Thus, if the initial ratio of the two enantiomers is 50:50, such as is present in a racemic mixture, and an enantiomeric enrichment sufficient to produce a final ratio of 70:30 is achieved, the ee with respect to the first enantiomer is 40%. However, if the final ratio is 90:10, the ee with respect to the first enantiomer is 80%. An ee of greater than 90% is preferred, an ee of greater than 95% is most preferred and an ee of greater than 99% is most especially preferred. Enantiomeric enrichment is readily determined by one of ordinary skill in the art using standard techniques and procedures, such as gas or high performance liquid chromatography with a chiral column. Choice of the appropriate chiral column, eluent and conditions necessary to effect separation of the enantiomeric pair is well within the knowledge of one of ordinary skill in the art. In addition, the specific stereoisomers and enantiomers of compounds of Formula I can be prepared by one of ordinary skill in the art utilizing well known techniques and processes, such as those disclosed by J. Jacques, et al., “Enantiomers, Racemates, and Resolutions,” John Wiley and Sons, Inc., 1981, and E. L. Eliel and S. H. Wilen, “Stereochemistry of Organic Compounds,” (Wiley-Interscience 1994), and European Patent Application No. EP-A-838448, published Apr. 29, 1998. Examples of resolutions include recrystallization techniques or chiral chromatography.

Some of the compounds of the present invention have one or more chiral centers and may exist in a variety of stereoisomeric configurations. As a consequence of these chiral centers, the compounds of the present invention occur as racemates, mixtures of enantiomers and as individual enantiomers, as well as diastereomers and mixtures of diastereomers. All such racemates, enantiomers, and diastereomers are within the scope of the present invention.

The terms “R” and “S” are used herein as commonly used in organic chemistry to denote specific configuration of a chiral center. The term “R” (rectus) refers to that configuration of a chiral center with a clockwise relationship of group priorities (highest to second lowest) when viewed along the bond toward the lowest priority group. The term “S” (sinister) refers to that configuration of a chiral center with a counterclockwise relationship of group priorities (highest to second lowest) when viewed along the bond toward the lowest priority group. The priority of groups is based upon their atomic number (in order of decreasing atomic number). A partial list of priorities and a discussion of stereochemistry is contained in “Nomenclature of Organic Compounds: Principles and Practice,” (J. H. Fletcher, et al., eds., 1974) at pages 103-120.

The designation [symbol] refers to a bond that protrudes forward out of the plane of the page. The designation [symbol] refers to a bond that protrudes backward out of the plane of the page. The designation [symbol] refers to a bond wherein the stereochemistry is not defined.

In general, the term “pharmaceutical” when used as an adjective means substantially non-toxic to living organisms. For example, the term “pharmaceutical salt” as used herein, refers to salts of the compounds of Formula I which are substantially non-toxic to living organisms. See, e.g., Berge, S. M, Bighley, L. D., and Monkhouse, D. C., “Pharmaceutical Salts, “J. Pharm. Sci., 66:1, 1977. Typical pharmaceutical salts include those salts prepared by reaction of the compounds of Formula I with an inorganic or organic acid or base. Such salts are known as acid addition or base addition salts respectively. These pharmaceutical salts frequently have enhanced solubility characteristics compared to the compound from which they are derived, and thus are often more amenable to formulation as liquids or emulsions.

The term “acid addition salt” refers to a salt of a compound of Formula I prepared by reaction of a compound of Formula I with a mineral or organic acid. For exemplification of pharmaceutical acid addition salts see, e.g., Berge, S. M, Bighley, L. D., and Monkhouse, D. C., J. Pharm. Sci., 66:1, 1977. Since compounds of this invention can be basic in nature, they accordingly react with any of a number of inorganic and organic acids to form pharmaceutical acid addition salts.

The pharmaceutical acid addition salts of the invention are typically formed by reacting the compound of Formula I with an equimolar or excess amount of acid. The reactants are generally combined in a mutual solvent such as diethylether, tetrahydrofuran, methanol, ethanol, isopropanol, benzene, and the like. The salts normally precipitate out of solution within about one hour to about ten days and can be isolated by filtration or other conventional methods.

Acids commonly employed to form acid addition salts are inorganic acids such as hydrochloric acid, hydrobromic acid, hydroiodic acid, sulfuric acid, phosphoric acid, and the like, and acids commonly employed to form such salts are inorganic acids such as hydrochloric acid, hydrobromic acid, hydroiodic acid, sulfuric acid, phosphoric acid, and the like, and organic acids, such as p-toluenesulfonic acid, methanesulfonic acid, oxalic acid, p-bromophenylsulfonic acid, carbonic acid, succinic acid, citric acid, benzoic acid, acetic acid and the like. Examples of such pharmaceutically acceptable salts thus are the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, β-hydroxybutyrate, glycollate, tartrate, methanesulfonate, propanesulfonate, naphthalene-1-sulfonate, naphthalene-2-sulfonate, mandelate and the like.

The term “base addition salt” refers to a salt of a compound of Formula I prepared by reaction of a compound of Formula I with a mineral or organic base. For exemplification of pharmaceutical base addition salts see, e.g., Berge, S. M, Bighley, L. D., and Monkhouse, D. C., J. Pharm. Sci., 66:1, 1977. This invention also contemplates pharmaceutical base addition salts of compounds of Formula I. The skilled artisan would appreciate that some compounds of Formula I may be acidic in nature and accordingly react with any of a number of inorganic and organic bases to form pharmaceutical base addition salts. Examples of pharmaceutical base addition salts are the ammonium, lithium, potassium, sodium, calcium, magnesium, methylamino, diethylamino, ethylene diamino, cyclohexylamino, and ethanolamino salts, and the like of a compound of Formula I.

The compounds of Formula I, when existing as a diastereomeric mixture, may be separated into diastereomeric pairs of enantiomers by, for example, fractional crystallization from a suitable solvent, for example methanol or ethyl acetate or a mixture thereof. The pair of enantiomers thus obtained may be separated into individual stereoisomers by conventional means, for example by the use of an optically active acid as a resolving agent. Alternatively, any enantiomer of a compound of the formula may be obtained by stereospecific synthesis using optically pure starting materials or reagents of known configuration or through enantioselective synthesis.

The compounds of Formula I can be prepared by one of ordinary skill in the art following a variety of procedures, some of which are illustrated in the procedures and schemes set forth below. The particular order of steps required to produce the compounds of Formula I is dependent upon the particular compound to being synthesized, the starting compound, and the relative liability of the substituted moieties. The reagents or starting materials are readily available to one of skill in the art, and to the extent not commercially available, are readily synthesized by one of ordinary skill in the art following standard procedures commonly employed in the art, along with the various procedures and schemes set forth below.

The following Preparations and Examples are provided to better elucidate the practice of the present invention and should not be interpreted in any way as to limit the scope of the same. Those skilled in the art will recognize that various modifications may be made while not departing from the spirit and scope of the invention. All publications mentioned in the specification are indicative of the level of those skilled in the art to which this invention pertains.

The terms and abbreviations used in the instant Preparations and Examples have their normal meanings unless otherwise designated. For example, as used herein, the following terms have the meanings indicated: “eq” refers to equivalents; “N” refers to normal or normality, “M” refers to molar or molarity, “g” refers to gram or grams, “mg” refers to milligrams; “L” refers to liters; “mL” refers to milliliters; “μL” refers to microliters; “mol” refers to moles; “mmol” refers to millimoles; “psi” refers to pounds per square inch; “min” refers to minutes; “h” or “hr” refers to hours; “° C.” refers to degrees Celsius; “TLC” refers to thin layer chromatography; “HPLC” refers to high performance liquid chromatography; “R_{f}” refers to retention factor; “Rₜ” refers to retention time; “δ” refers to part per million down-field from tetramethylsilane; “MS” refers to mass spectrometry, Observed Mass indicates (M+1) unless indicated otherwise. “MS(FD)” refers to field desorption mass spectrometry, “MS(IS)” refers to ion spray mass spectrometry, “MS(FIA)” refers to flow injection analysis mass spectrometry, “MS(FAB)” refers to fast atom bombardment mass spectrometry, “MS(EI)” refers to electron impact mass spectrometry, “MS(ES)” refers to electron spray mass spectrometry, “UV” refers to ultraviolet spectrometry, “¹H NMR” refers to proton nuclear magnetic resonance spectrometry. In addition, “IR” refers to infrared spectrometry, and the absorption maxima listed for the IR spectra are only those of interest and not all of the maxima observed. “RT” refers to room temperature. 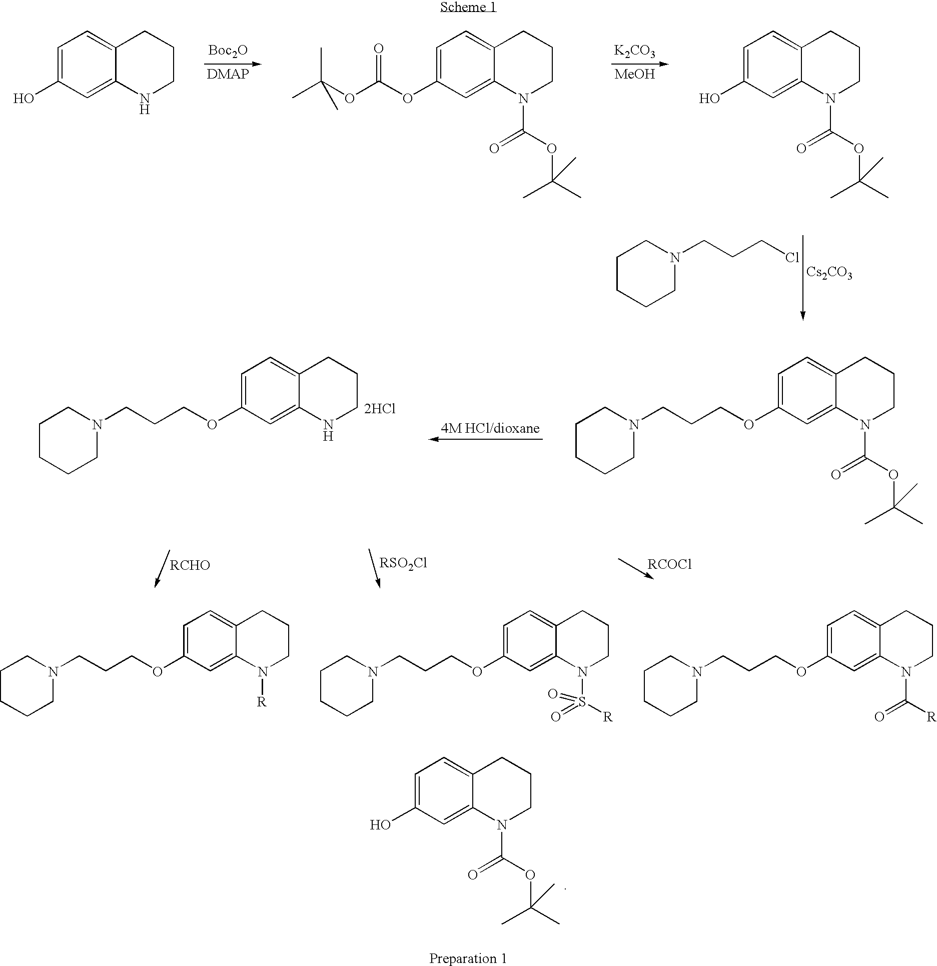
7-Hydroxy-3,4-dihydro-2H-quinoline-1-carboxylic acid tert-butyl ester 1,2,3,4-Tetrahydro-quinolin-7-ol (CAS Registry Number 58196-33-1) (6.53 g, 43.8 mmol) is treated with di-tert-butyl dicarbonate (57.3 g, 262.6 mmol) and DMAP (0.53 g, 4.38 mmol) in THF (200 mL) and the mixture is heated at reflux overnight. An additional quantity of di-tert-butyl dicarbonate (25.0 g, 131.2 mmol) and DMAP (0.53 g, 4.38 mmol) is added and the reaction stirred at room temperature overnight. The solvent is removed, and the crude material purified by flash chromatography (5-35% EtOAc/hexanes) to provide 7-tert-Butoxycarbonyloxy-3,4-dihydro-2H-quinoline-1-carboxylic acid tert-butyl ester (8.75 g, 57%).

7-tert-Butoxycarbonyloxy-3,4-dihydro-2H-quinoline-1-carboxylic acid tert-butyl ester (8.75 g, 25.0 mmol) is dissolved in methanol (150 mL) and K₂CO₃ (6.9 g, 50.1 mmol) is added. This mixture is allowed to stir at room temperature overnight. The solid K₂CO₃ is filtered off, and the solvent evaporated. The crude mixture is taken up in EtOAc, and washed with water (2×), brine (1×), dried over Na₂SO₄ and the organic layer evaporated to give a crude oil. Flash chromatography (Biotage, 20% EtOAc/hexanes) gives the desired material (5.39 g, 86.3%).

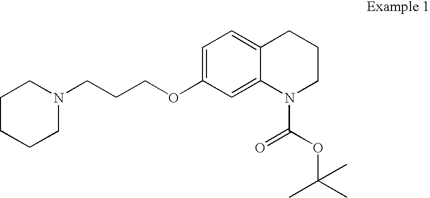

### 7-(3-Piperidin-1-yl-propoxy)-3,4-dihydro-2H-quinoline-1-carboxylic acid tert-butyl ester

Procedure A: A 225 mL dioxane solution of 7-hydroxy-3,4-dihydro-2H-quinoline-1-carboxylic acid tert-butyl ester (5.0 g, 20 mmol) is stirred under N₂ as Cs₂CO₃ (13.1 g, 40.1 mmol), KI (0.33 g, 2 mmol), then N-(3-chloropropyl)piperidine (3.9 g, 24 mmol) are added in succession. The reaction mixture is heated at 90° C. for 10 hours, cooled, filtered, and concentrated to give the crude product. Purification by chromatography (SiO₂; 0-10% MeOH/CH₂Cl₂/1% NH₄OH gradient) gives the product. (7.3 g, 97% yld). MS (ES+)375.3(M+H)⁺. Calculated for C₂₂H₃₄N₂O₃: C, 70.5; H, 9.15; N, 7.48. Found: C, 70.2; H, 8.91; N, 7.42.

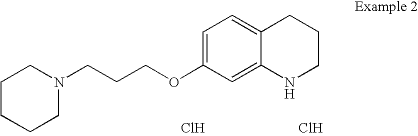

### 7-(3-Piperidin-1-yl-propoxy)-1,2,3,4-tetrahydro-quinoline hydrochloride

Procedure B: A 45 mL CH₂Cl₂ solution of 7-(3-Piperidin-1-yl-propoxy)-3,4-dihydro-2H-quinoline-1-carboxylic acid tert-butyl ester (1.6 g, 6.58 mmol) is stirred under N₂ at 0-10° C. as 4N HCl/dioxane (4.9 mL, 19.7 mmol) is added dropwise. After the addition is complete, reaction mixture is stirred at this temperature for 30-60 min, then allowed to warm to room temperature. A white precipitate forms and dry MeOH is added until clear solution is obtained. Additional 4N HCl/dioxane (4.9 mL, 19.7 mmol) is added dropwise. After the addition is complete, reaction mixture is stirred at room temperature. Reaction is followed by TLC (SiO₂ plate, CH₃Cl/MeOH/NH₄OH; 25/5/1) until starting material consumed (4-5 h). Reaction mixture is concentrated, dissolved in dry MeOH, concentrated, triturated in Et₂O, filtered, and dried in vacuo to give the HCl salt (1.2 g, 54% yld.) MS(ES+) 275.2(M+H)⁺free base. FTIR (CHCl₃)(cm⁻¹): 2960, 2638, 2466, 1630, 1598, 1516, 1461, 1282, 1242.

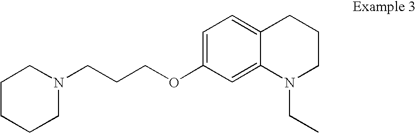

### 1-Ethyl-7-(3-piperidin-1-yl-propoxy)-1,2,3,4-tetrahydro-quinoline

Procedure C: A 14 mL CH₂Cl₂/MeOH (9:1) solution of 7-(3-piperidin-1-yl-propoxy)-1,2,3,4-tetrahydro-quinoline dihydrochloride (0.3 g, 0.86 mmol) is stirred under N₂, the MP-CNBH₃ resin (1.1 g, 2.6 mmol) added, the acetaldehyde (0.23 g, 5.2 mmol) added, the pH is adjusted to −4 with glacial AcOH and reaction mixture stirred at room temperature for 18-20 hours. The reaction mixture is filtered and the resin beads washed twice alternately with MeOH, then CH₂Cl₂. The filtrate is concentrated and the residue is purified by chromatography (SCX-MeOH wash, elute 2M NH₃/MeOH; then (SiO₂; 0-10% MeOH/CH₂Cl₂/1% NH₄OH gradient) to give the pure free base MS(ES+) 303.3(M+H)⁺. 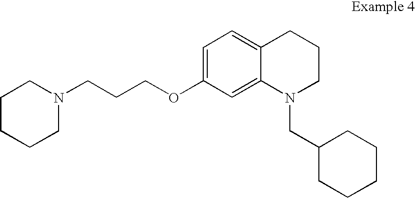
1-Cyclohexylmethyl-7-(3-piperidin-1-yl-propoxy)-1,2,3,4-tetrahydro-quinoline: 1-Cyclohexylmethyl-7-(3-piperidin-1-yl-propoxy)-1,2,3,4-tetrahydro-quinoline is prepared from 7-(3-Piperidin-1-yl-propoxy)-1,2,3,4-tetrahydro-quinoline dihydrochloride (0.3 g, 0.86 mmol) and cyclohexane carboxyaldehyde (0.62 mL, 5.2 mmol) in a manner substantially analogous to Procedure C (See herein Example 3). MS(ES+) 371.4(M+H)⁺.

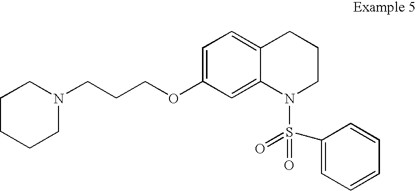

### 1-Benzenesulfonyl-7-(3-piperidin-1-yl-propoxy)-1,2,3,4-tetrahydro-quinoline

Procedure D: A 5 mL CH₂Cl₂ solution of 7-(3-piperidin-1-yl-propoxy)-1,2,3,4-tetrahydro-quinoline dihydrochloride (150 mg, 0.5 mmol) and NEt₃ (0.27 mL, 3.8 mmol) is stirred under N₂, benzenesulfonyl chloride (0.12 mL, 0.94 mmol) is added, and reaction is stirred at room temperature for 72 hours. Reaction mixture is diluted with EtOAc, washed with saturated aqueous Na₂CO₃, and the aqueous layer back-extracted with EtOAc. The EtOAc extracts are combined, dried (Na₂SO₄), and concentrated. The residue is purified by chromatography (SCX-MeOH wash, elute 2M NH₃/MeOH, then SiO₂; 0-6% MeOH/CH₂Cl₂/1% Nh₄OH gradient) to give the free base (60 mg, 33% yld). MS(ES+) 415.3(M+H)⁺free base. 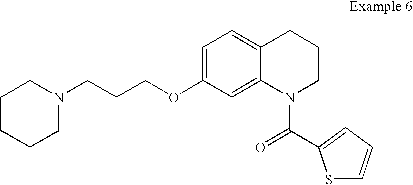
[7-(3-Piperidin-1-yl-propoxy)-3,4-dihydro-2H-quinolin-1-yl]-thiophen-2-yl-methanone: A 5 mL CH₂Cl₂ solution of 7-(3-piperidin-1-yl-propoxy)-1,2,3,4-tetrahydro-quinoline dihydrochloride (150 mg, 0.5 mmol) and NEt₃ (0.27 mL, 2.0 mmol) is stirred under N₂, a 1 mL CH₂Cl₂ solution of 2-thiophene carbonyl chloride (0.125 mL, 0.80 mmol) is added, and reaction is stirred at room temperature for 72 hours. Reaction mixture is diluted with EtOAc, washed with saturated aqueous Na₂CO₃, and the aqueous layer extracted with EtOAc. The EtOAc extracts are combined, dried (Na₂SO₄), and concentrated. The residue is purified by chromatography (SCX-MeOH wash, elute 2M NH₃/MeOH; then SiO₂; 0-10% MeOH/CH₂Cl₂/1% NH₄OH gradient) to give the free base (100 mg, 55% yld). MS(ES+)385.3(M+H)⁺free base.

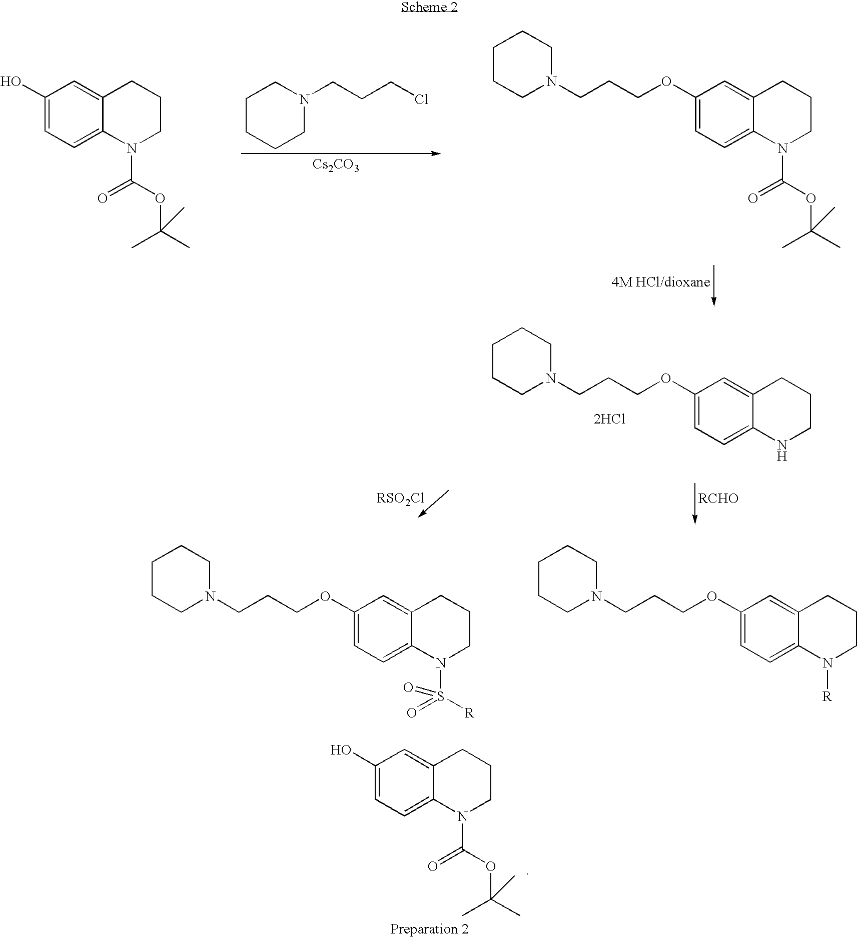

### 6-Hydroxy-3,4-dihydro-2H-quinoline-1-carboxylic acid tert-butyl ester

Procedure E: 1,2,3,4-tetrahydro-quinolin-6-ol (CAS Registry Number 3373-00-0) (25 g, 172.9 mmol) is treated with di-tert-butyl dicarbonate (56.6 g, 259.4 mmol) in a solvent mixture of dioxane (200 mL) and 1N NaOH (200 mL), and reaction mixture allowed to stir at room temperature overnight. The layers are separated, and the aqueous layer is washed with ethyl acetate (2×). The organic layers are combined and washed with brine (1×), dried over Na₂SO₄ and the organic layer evaporated to give a crude oil. Flash chromatography (Biotage, 10% EtOAc/hexanes) gives the desired material (33.0 g, 76.7% yld). 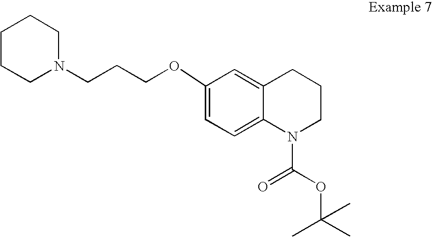
6-(3-Piperidin-1-yl-propoxy)-3,4-dihydro-2H-quinoline-1-carboxylic acid tert-butyl ester: 6-(3-Piperidin-1-yl-propoxy)-3,4-dihydro-2H-quinoline-1-carboxylic acid tert-butyl ester is prepared 6-hydroxy-3,4-dihydro-2H-quinoline-1-carboxylic acid tert-butyl ester (5.0 g, 20.0 mmol) in a manner substantially analogous to Procedure A (See herein Example 1) in 97% yield. MS(ES+) 375.3(M+H)⁺. FTIR (CHCl₃)(cm⁻¹): 3010, 2939, 1683, 1501, 1471, 1369, 1270, 1255, 1164, 1141, 1133. 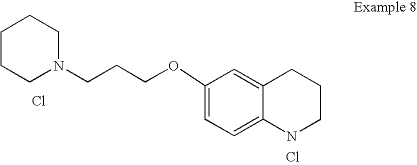
6-(3-Piperidin-1-yl-propoxy)-1,2,3,4-tetrahydro-quinoline dihydrochloride: 6-(3-Piperidin-1-yl-propoxy)-1,2,3,4-tetrahydro-quinoline dihydrochloride is prepared from 6-(3-piperidin-1-yl-propoxy)-3,4-dihydro-2H-quinoline-1-carboxylic acid tert-butyl ester (2.73 g, 10.9 mmol) in a manner substantially analogous to Procedure B (See herein Example 2) in 73% yield. MS(ES+)275.2(M+H)+free base. FTIR (CHCl₃)(cm⁻¹): 2936, 2887, 2643, 2512, 1626, 1598, 1511, 1460, 1275, 1175. 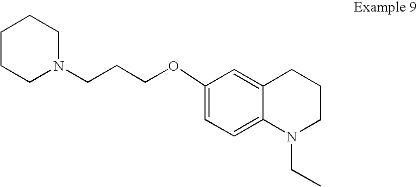
1-Ethyl-6-(3-piperidin-1-yl-propoxy)-1,2,3,4-tetrahydro-quinoline: 1-Ethyl-6-(3-piperidin-1-yl-propoxy)-1,2,3,4-tetrahydro-quinoline is prepared from 6-(3-piperidin-1-yl-propoxy)-1,2,3,4-tetrahydro-quinoline dihydrochloride in a manner substantially analogous to Procedure C (See herein Example 3). MS(ES+) 303.3(M+1)⁺. 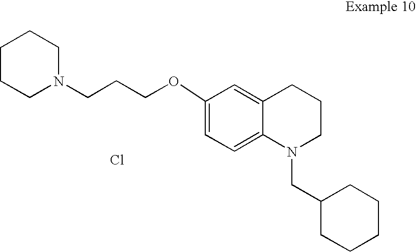
1-Cyclohexylmethyl-6-(3-piperidin-1-yl-propoxy)-1,2,3,4-tetrahydro-quinoline hydrochloride: 1-cyclohexylmethyl-6-(3-piperidin-1-yl-propoxy)-1,2,3,4-tetrahydro-quinoline is prepared from 6-(3-Piperidin-1-yl-propoxy)-1,2,3,4-tetrahydro-quinoline dihydrochloride (346 mg, 1 mmol), cyclohexanecarboxaldehyde (0.73 mL, 6 mmol), and MP-CNBH₃ resin (1.6 g, 4 mmol) in a manner substantially analogous to Procedure C (See herein Example 3) to give the free base. Procedure F: A 5 mL THF/MeOH (1:1) solution of the free base (0.28 g, 0.75 mmol) is stirred under N₂ at 0-10° C. as 1N HCl/Et₂O (1.6 mL, 1.6 mmol) is added dropwise. After the addition is complete, reaction mixture is allowed to warm to room temperature, then reaction mixture is concentrated, dissolved in dry MeOH, concentrated, triturated in Et₂O, filtered, and dried in vacuo to give the product as the HCl salt (237 mg, 58% yld). MS(ES+) 371.3(M+H)⁺. 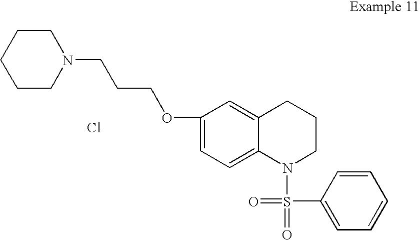
1-Benzenesulfonyl-6-(3-piperidin-1-yl-propoxy)-1,2,3,4-tetrahydro-quinoline hydrochloride: 1-Benzenesulfonyl-6-(3-piperidin-1-yl-propoxy)-1,2,3,4-tetrahydro-quinoline hydrochloride is prepared from 6-(3-piperidin-1-yl-propoxy)-1,2,3,4-tetrahydro-quinoline dihydrochloride (260 mg, 0.75 mmol), NEt₃ (0.35 mL, 2.5 mmol), and benzenesulfonyl chloride (0.12 mL, 0.94 mmol) via a procedure substantially analogous to Procedure D (See herein Example 5) except that an additional SCX column purification step is performed to give the free base product. A 5 mL dry MeOH solution of the free base (189 mg, 0.56 mmol) is stirred with 1N HCl/Et₂O (0.90 mL, 0.9 mmol) for 5 minutes, concentrated, triturated with Et₂O, filtered, and dried in vacuo to give the HCl salt (189 mg, 56% yld). MS(ES+) 415.2 (M+H)⁺ free base. 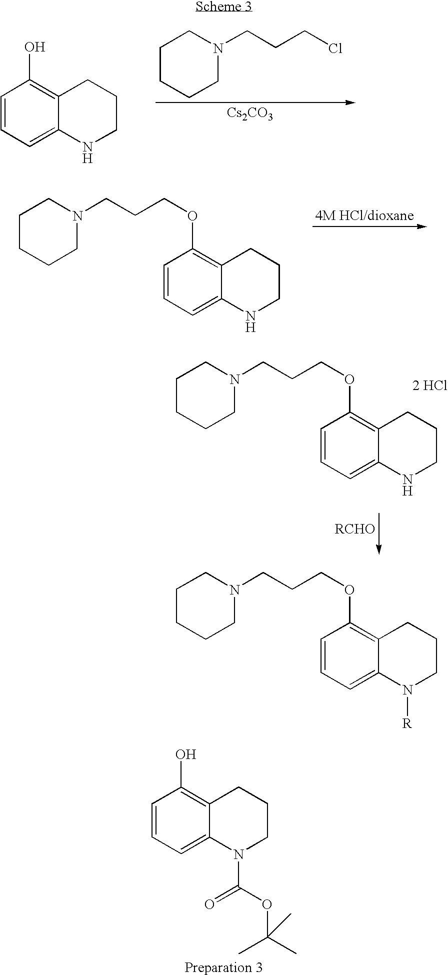
5-Hydroxy-3,4-dihydro-2H-quinoline-1-carboxylic acid tert-butyl ester: 5-Hydroxy-3,4-dihydro-2H-quinoline-1-carboxylic acid tert-butyl ester is prepared from 1,2,3,4-tetrahydro-quinolin-6-ol (CAS Registry Number 61468-43-7) via a procedure substantially analogous for the preparation of 7-hydroxy-3,4-dihydro-2H-quinoline-1-carboxylic acid tert-butyl ester (See herein Preparation 1). MS(ES+) 250.1(M+H)⁺. 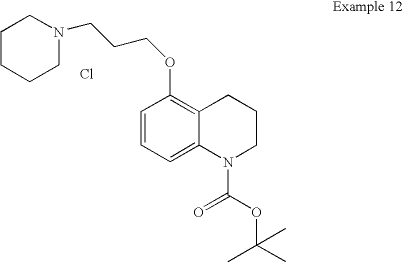
5-(3-Piperidin-1-yl-propoxy)-3,4-dihydro-2H-quinoline-1-carboxylic acid tert-butyl ester hydrochloride: 5-(3-Piperidin-1-yl-propoxy)-3,4-dihydro-2H-quinoline-1-carboxylic acid tert-butyl ester is prepared from 5-hydroxy-3,4-dihydro-2H-quinoline-1-carboxylic acid tert-butyl ester (660 mg, 2.6 mmol) in a manner substantially analogous to Procedure A (See herein Example 1) in 23% yield. The free base (21 mg, 0.05 mmol) is converted to the HCl salt (20 mg, 49 mmol) with 1N HCl/Et₂O (0.08 mL, 0.08 mmol). MS(ES+) 375.4(M+H)⁺free base. 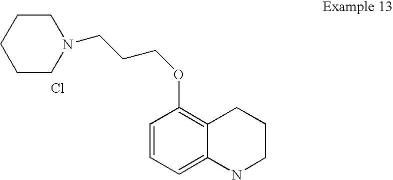
5-(3-Piperidin-1-yl-propoxy)-1,2,3,4-tetrahydro-quinoline hydrochloride: 5-(3-Piperidin-1-yl-propoxy)-1,2,3,4-tetrahydro-quinoline hydrochloride is prepared from 5-(3-piperidin-1-yl-propoxy)-3,4-dihydro-2H-quinoline-1-carboxylic acid tert-butyl ester (180 mg, 0.48 mmol) in a manner substantially analogous to Procedure B (See herein Example 2) in 100% yield. MS(ES+) 275.2(M+H)⁺free base. 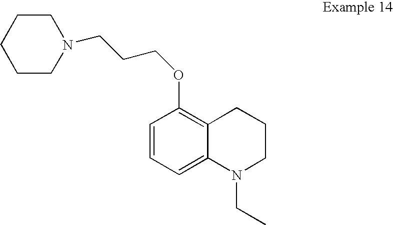
1-Ethyl-5-(3-piperidin-1-yl-propoxy)-1,2,3,4-tetrahydro-quinoline: 1-Ethyl-5-(3-piperidin-1-yl-propoxy)-1,2,3,4-tetrahydro-quinoline prepared from 5-(3-piperidin-1-yl-propoxy)-1,2,3,4-tetrahydro-quinoline hydrochloride (78 mg, 0.25 mmol), acetaldehyde (0.2 mL, 3.5 mmol), and MP-CNBH₃ resin (400 mg, 1 mmol) in a manner substantially analogous to Procedure C (See herein Example 3) to give the product as an amber oil (58 mg, 77% yld). MS(ES+) 303.3(M+H)⁺. 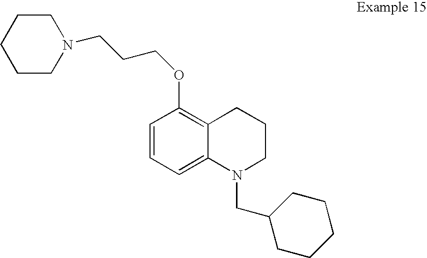
1-Cyclohexylmethyl-5-(3-piperidin-1-yl-propoxy)-1,2,3,4-tetrahydro-quinoline: 1-Cyclohexylmethyl-5-(3-piperidin-1-yl-propoxy)-1,2,3,4-tetrahydro-quinoline is prepared from 5-(3-piperidin-1-yl-propoxy)-1,2,3,4-tetrahydro-quinoline hydrochloride (31 mg, 0.1 mmol), cyclohexane carboxaldehyde (0.1 mL, 0.65 mmol), and MP-CNBH₃ resin (210 mg, 0.5 mmol) in a manner substantially analogous to Procedure C (See herein Example 3) to give the product as an amber oil (20 mg, 54% yld). MS(ES+) 371.4(M+H)⁺. 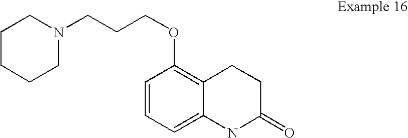
5-(3-Piperidin-1-yl-propoxy)-3,4-dihydro-1H-quinolin-2-one is prepared from 5-hydroxy-3,4-dihydro-1H-quinolin-2-one (CAS Registry Number 30389-33-4) (0.5 g, 3.06 mmol) in a manner substantially analogous to Procedure A (See herein Example 1) to give the title compound as a white solid (0.218 g, 25%). MS (ES+) 289.1

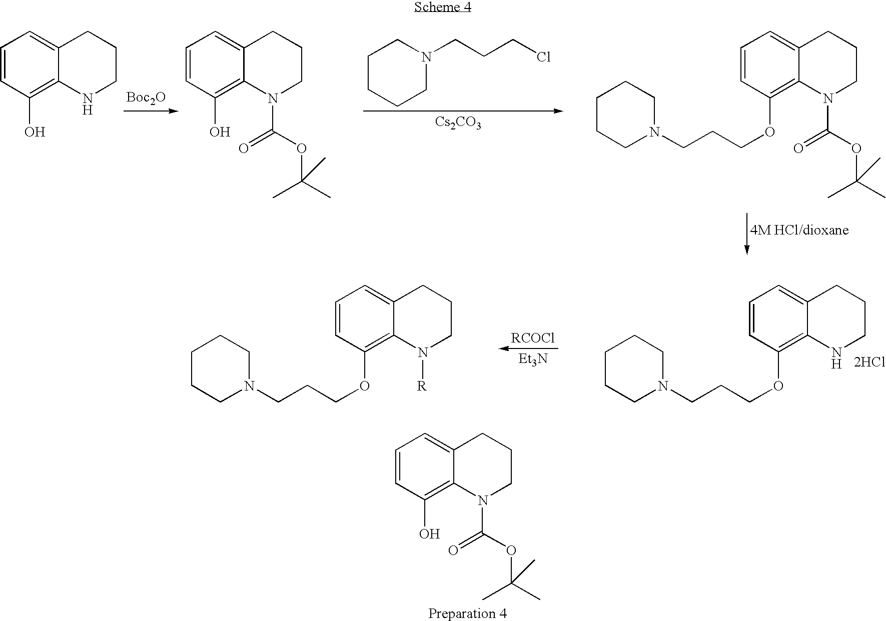

### 8-Hydroxy-3,4-dihydro-2H-quinoline-1-carboxylic acid tert-butyl ester

Procedure G: 1,2,3,4-Tetrahydro-quinolin-8-ol (CAS Registry Number 6640-50-2) (25.8 g, 172.9 mmol) is treated with di-tert-butyl dicarbonate (56.6 g, 259.4 mmol) in a solvent mixture of dioxane (200 mL) and 1N NaOH (200 mL), and reaction mixture allowed to stir at room temperature overnight. The layers are separated, and the aqueous layer is washed with ethyl acetate (2×). The organic layers are combined and washed with brine (1×), dried over Na₂SO₄ and the organic layer evaporated to give a crude oil. Flash chromatography (Biotage, 10% EtOAc/hexanes) gives the desired material (36.5 g, 84.7%). Calculated for C₁₄H₁₉NO₃: C, 67.44; H, 7.68; N, 5.62. Found: C, 67.91; H, 7.92; N, 5.76. 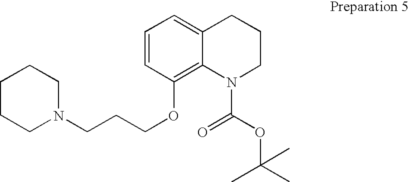
8-(3-Piperidin-1-yl-propoxy)-3,4-dihydro-2H-quinoline-1-carboxylic acid tert-butyl ester: 8-(3-Piperidin-1-yl-propoxy)-3,4-dihydro-2H-quinoline-1-carboxylic acid tert-butyl ester is prepared from 8-Hydroxy-3,4-dihydro-2H-quinoline-1-carboxylic acid tert-butyl ester (5.0 g, 20.0 mmol) in a manner substantially analogous to Procedure A (See herein Example 1) in 49% yield. HRMS 375.2644(M+It)⁺. FTIR (CHCl₃)(cm⁻¹): 3008, 2939, 1687, 1488, 1470, 1367, 1272, 1255, 1164, 1081.

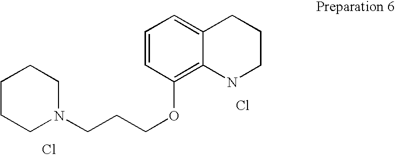

### 8-(3-Piperidin-1-yl-propoxy)-1,2,3,4-tetrahydro-quinoline dihydrochloride

8-(3-Piperidin-1-yl-propoxy)-1,2,3,4-tetrahydro-quinoline dihydrochloride is prepared from 8-(3-Piperidin-1-yl-propoxy)-3,4-dihydro-2H-quinoline-1-carboxylic acid tert-butyl ester (3.7 g, 9.77 mmol) in a manner substantially analogous to Procedure B (See herein Example 2) in 71% yield. MS(ES+) 275.2(M+H)⁺free base. FTIR (CHCl₃)(cm⁻¹): 2961, 2630, 2472, 1625, 1600, 1469, 1282, 1239, 1107. 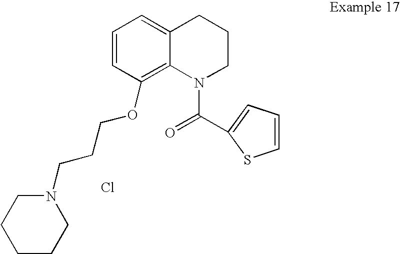
[8-(3-Piperidin-1-yl-propoxy)-3,4-dihydro-2H-quinolin-1-yl]-thiophen-2-yl-methanone: A 5 mL CH₂Cl₂ solution of 8-(3-piperidin-1-yl-propoxy)-1,2,3,4-tetrahydro-quinoline dihydrochloride (175 mg, 0.5 mmol) and NEt₃ (0.25 mL, 1.7 mmol) is stirred under N₂, a 1 mL CH₂Cl₂ solution of 2-thiophene carbonyl chloride (0.068 mL, 0.63 mmol) is added, and reaction is stirred at room temperature for 18 hours. Reaction appears to be incomplete, so additional NEt₃ (0.25 mL, 1.7 mmol) and thiophene carbonyl chloride (0.068 mL, 0.63 mmol) are added, reaction stirred 4 h at 35° C., then 18 h at room temperature. Reaction mixture is quenched with MeOH, concentrated and the residue is purified by chromatography (SCX-MeOH wash, elute 2M NH₃/MeOH; then SiO₂; 0-10% MeOH/CH₂Cl₂/1% NH₄OH gradient) to give the free base (122 mg). A 3 mL dry MeOH solution of the free base (122 mg, 0.32 mmol) is stirred with 1N HCl/Et₂O (0.45 mL, 0.45 mmol) for 5 minutes, concentrated, triturated with Et₂O, filtered, and dried in vacuo to give the HCl salt (125 mg, 60% yld). MS(ES+)385.3(M+H)⁺free base. 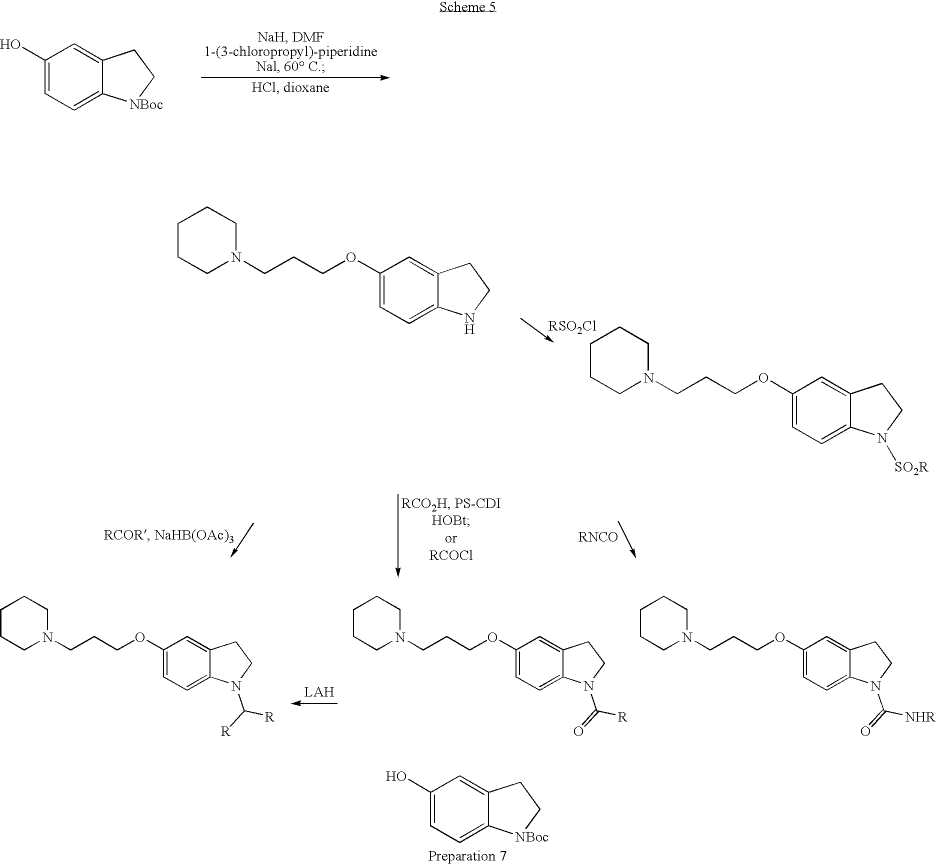
1-(tert-Butoxycarbonyl)-5-hydroxyindoline; prepared by the method described in Stark, et al., J. Org. Chem. 2000, 65, 3227.

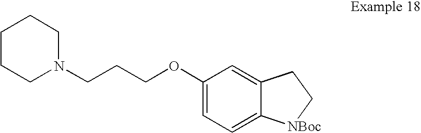

### 5-(3-Piperidin-1-yl-propoxy)-2,3-dihydro-indole-1-carboxylic acid tert-butyl ester

Procedure H: To a stirred solution of 1-(tert-butoxycarbonyl)-5-hydroxyindoline (3.29 g, 13.98 mmol) in dry dimethylformamide (DMP) (30 mL) at room temperature under N₂, was added sodium hydride (60% dispersion, 0.67 g, 16.75 mmol) portion wise. The mixture was stirred for 15 minutes, and 1-(3-chloropropyl)-piperidine (2.8 mL, ˜17.4 mmol) was added, followed by sodium iodide (2.0 g, 13.3 mmol). After heating for 4 hours at 70° C., the reaction mixture was cooled to room temperature, poured into water, extracted three times with ethyl acetate, dried over anhydrous potassium carbonate and concentrated in vacuo, to provide quantitatively, 5-(3-piperidin-1-yl-propoxy)-2,3-dihydro-indole-1-carboxylic acid tert-butyl ester. A portion was purified by flash chromatography on silica gel (30:1 dichloromethane(DCM)/7N NH₃ in methanol), yielding a white solid. ¹H NMR (CDCl₃) δ 7.72 (bs, 0.6H), 7.33 (bs, 0.411), 6.73 (bs, 1H), 6.69 (bd, 1H), 3.96 (t, 4H), 3.04 (t, 2H), 2.49 (t, 2), 2.42 (bs, 4H), 1.97 (qt, 2H), 1.55-1.64 (m, 13H), 1.45 (bm, 2H); MS (APCI), M+H: 361 (100%). 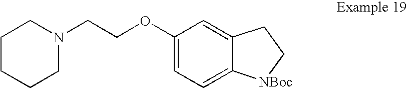
5-(2-Piperidin-1-yl-ethoxy)-2,3-dihydro-indole-1-carboxylic acid tert-butyl ester; prepared in quantitative yield from 1-(tert-butoxycarbonyl)-5-hydroxyindoline (2.0 g, 8.5 mmol) and 1-(2-chloroethyl)-piperidine (1.37 g, 9.3 mmol) by the method of Procedure H (See herein Example 18). A portion was purified by flash chromatography on silica gel (20:1 DCM/7N NH₃ in methanol) to give the title compound as an off-white solid. ¹H NMR (CDCl₃) δ 7.73 (bs, 0.5H), 7.32 (bs, 0.5H), 6.74 (s, 1H), 6.70 (d, 1H), 4.09 (t, 2H), 3.97 (bs, 2H), 3.05 (t, 2H), 2.79 (t, 2H), 2.55 (bs, 4H), 1.64 (m, 4H), 1.54 (bs, 9H) 1.47 (m, 2H); MS (APCI), M+H: 347 (100%). 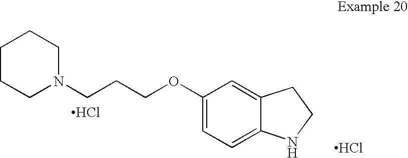
5-(3-Piperidin-1-yl-propoxy)-2,3-dihydro-1H-indole dihydrochloride; prepared as a hygroscopic white solid (200 mg, 90%) from 5-(3-piperidin-1-yl-propoxy)-2,3-dihydro-indole-1-carboxylic acid tert-butyl ester (240 mg, 0.667 mmol) by the method of Procedure B (See herein Example 2). ¹H NMR (free base in CDCl₃) δ 6.76 (s, 1H), 6.58 (m, 2H), 3.92 (t, 2H), 3.53 (t, 2H), 3.00 (t, 2H), 2.46 (t, 2H), 2.40 (bs, 4H), 1.93 (m, 2H), 1.59 (m, 4H) 1.44 (m, 2H); MS (APCI), M+H: 261 (100%). 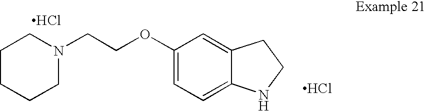
5-(2-Piperidin-1-yl-ethoxy)-2,3-dihydro-1H-indole dihydrochloride; prepared as a tan solid (2.7 g, 100% yield) from 5-(2-piperidin-1-yl-ethoxy)-2,3-dihydro-indole-1-carboxylic acid tert-butyl ester (2.9 g, 8.4 mmol) by the method of Procedure B (See herein Example 2). A portion was free based (Silicycle triamine-3/DCM/methanol/catalytic triethylamine), flash chromatographed on silica gel (20:1 DCM/7N NH₃ in methanol) and converted to the dihydrochloride (2M HCl in ether/DCM). ¹H NMR (free base in CDCl₃) δ 6.76 (s, 1H), 6.60 (dd, 1H), 6.56 (d, 1H), 4.03 (t, 2H), 3.52 (t, 2H), 2.99 (t, 2H), 2.73 (t, 2H), 2.49 (bt, 4H), 1.93 (m, 2H), 1.59 (m, 4H) 1.44 (m, 2H); MS (APCI), M+H: 247 (100%). 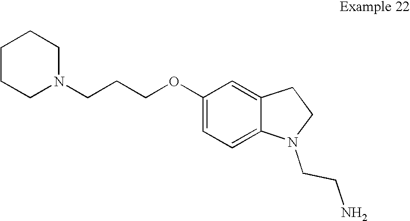
2-[5-(3-Piperidin-1-yl-propoxy)-2,3-dihydro-indol-1-yl]-ethylamine; prepared as a brown, waxy solid (8 mg, 88%) from {2-[5-(3-piperidin-1-yl-propoxy)-2;3-dihydro-indol-1-yl]-ethyl}-carbamic acid tert-butyl ester (12 mg, 0.03 mmol) according to the method of Procedure B (See herein Example 2). The trihydrochloride salt was free based (Argonaut PS-DIEA/catalytic triethylamine/DCM) to provide the title compound. ¹H NMR (CDCl₃) δ 6.70 (bs, 1H), 6.60 (bd, 1H), 6.47 (bd, 1H), 3.94 (t, 2H), 3.29 (t, 2H), 3.12 (t, 2H), 3.10 (t, 2H), 2.98 (t, 2H), 2.93 (t, 2H), 2.83 (bt, 2H), 2.79 (bs, 4H), 2.17 (bm, 2H), 1.85 (bm, 4H), 1.56 (bs, 2H); MS (ESI), M+H: 304 (100%).

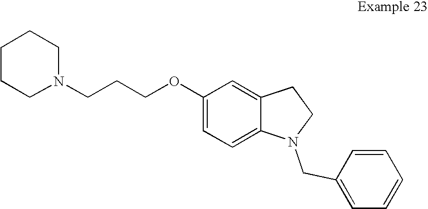

### 1-Benzyl-5-(3-piperidin-1-yl-propoxy)-2,3-dihydro-1H-indole

Procedure J: To a stirred solution of 5-(3-piperidin-1-yl-propoxy)-2,3-dihydro-1H-indole dihydrochloride (53 mg, 0.16 mmol) in 10:1 dichloroethane(DCE)/methanol (2.2 mL) containing acetic acid (0.1 equivalent) at room temperature under N₂, was added benzaldehyde (35 μl, 0.325 mmol). After 15 minutes, sodium triacetoxyborohydride (70 mg, 0.33 mmol) was added. Stirring was continued for 30 minutes (or until starting material was consumed by TLC) and the mixture was loaded directly onto a Varian SCX column (10 g). The column was washed with DCM and methanol, and the desired compound was then eluted with a 7N NH₃ in methanol, to provide 1-benzyl-5-(3-piperidin-1-yl-propoxy)-2,3-dihydro-1H-indole as a waxy white solid (53 mg 94%). ¹H NMR (CDCl₃) δ 7.31-7.38 (m, 4H), 7.26 (m, 1H), 6.74 (d, 1H), 6.59 (dd, 1H), 6.41 (d, 1H), 4.16 (s, 2H), 3.93 (t, 2H), 3.23 (t, 2H), 2.91 (t, 2H), 2.68 (bm, 4H), 2.62 (bm, 2H), 2.07 (bm, 2H), 1.73 (bm, 4H), 1.49 (bs, 2H); MS (APCI), M+H: 351 (100%). 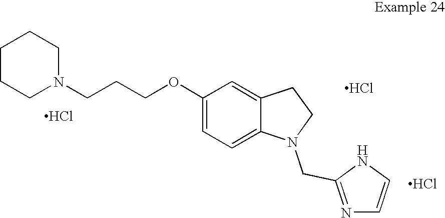
1-(1H-Imidazol-2-ylmethyl)-5-(3-piperidin-1-yl-propoxy)-2,3-dihydro-1H-indole, trihydrochloride; prepared from 5-(3-piperidin-1-yl-propoxy)-2,3-dihydro-1H-indole dihydrochloride (30 mg, 0.09 mmol) and 2-imidazolecarboxaldehyde (17 mg, 0.18 mmol) by the method of Procedure J (See herein Example 23). The free base was converted to the trihydrochloride with 2M HCl in ether/DCM, furnishing the title compound as a hygroscopic tan solid (26 mg, 64%). ¹H NMR (free base in CDCl₃) δ 7.01 (s, 2H), 6.73 (d, 1H), 6.56 (dd, 1H), 6.29 (dd, 1H), 4.25 (s, 2H), 3.91 (t, 2H), 3.29 (t, 2H), 2.90 (t, 2H), 2.64 (t, 2H), 2.60 (bs, 4H), 2.04 (m, 2H), 1.72 (m, 4H), 1.50 (m, 2H); MS (APCI), M+H: 341 (100%). 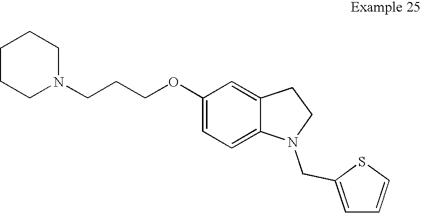
5-(3-Piperidin-1-yl-propoxy)-1-thiophen-2-ylmethyl-2,3-dihydro-1H-indole; prepared from 5-(3-piperidin-1-yl-propoxy)-2,3-dihydro-1H-indole dihydrochloride (30 mg, 0.09 mmol) and 2-thiophenecarboxaldehyde (17 μL, 0.18 mmol) by the method of Procedure J (See herein Example 23). The crude material was passed through a short plug of silica gel (20:1 DCM/7N NH₃ in methanol) to give the title compound as a brown oil (25 mg, 78%). ¹H NMR (CDCl₃) δ 7.21 (dd, 1H), 6.93-6.98 (m, 2H), 6.74 (d, 1H), 6.63 (dd, 1H), 6.49 (d, 1H), 4.37 (s, 2H), 3.92 (t, 2H), 3.27 (t, 2H), 2.90 (t, 2H), 2.47 (t, 2H), 2.41 (bm, 4H), 1.94 (m, 2H), 1.60 (m, 4H), 1.44 (m, 2H); MS (APCI), M+H: 357 (100%). 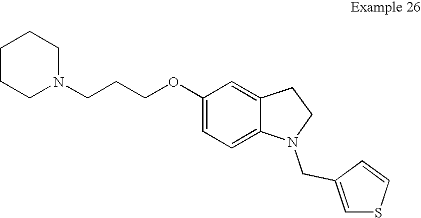
5-(3-Piperidin-1-yl-propoxy)-1-thiophen-3-ylmethyl-2,3-dihydro-1H-indole; prepared from 5-(3-piperidin-1-yl-propoxy)-2,3-dihydro-1H-indole dihydrochloride (30 mg, 0.09 mmol) and 3-thiophenecarboxaldehyde (17 μL, 0.18 mmol) by the method of Procedure J (See herein Example 23). The crude material was passed through a short plug of silica gel (20:1 DCM/7N NH₃ in methanol) to give the title compound as a brown oil (31 mg, 96%). ¹H NMR (CDCl₃) δ 7.28 (dd, 1”), 7.15 (m, 1”), 7.07 (dd, 1H), 6.74 (d, 1H), 6.62 (dd, 1H), 6.45 (d, 1H), 4.18 (s, 2H), 3.92 (t, 2H), 3.22 (t, 2H), 2.89 (t, 2H), 2.48 (t, 2H), 2.41 (bm, 4H), 1.95 (m, 2H), 1.60 (m, 4H), 1.45 (m, 2H); MS (APCI), M+H: 357 (100%). 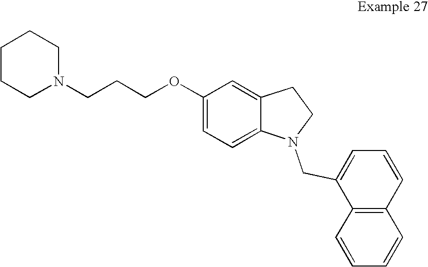
1-Naphthalen-1-ylmethyl-5-(3-piperidin-1-yl-propoxy)-2,3-dihydro-1H-indole; prepared from 5-(3-piperidin-1-yl-propoxy)-2,3-dihydro-1H-indole dihydrochloride (30 mg, 0.09 mmol) and 1-napthaldehyde (25 μL, 0.18 mmol) by the method of Procedure J (See herein Example 23). The crude material was passed through a short plug of silica gel (20:1 DCM/7N NH₃ in methanol) to give the title compound as a waxy, tan solid (32 mg, 89%). ¹H NMR (CDCl₃) δ 8.18 (m, 1H), 7.87 (m, 1H), 7.80 (d, 1H), 7.48-7.53 (m, 3H), 7.42 (t, 1H), 6.78 (d, 1H), 6.66 (dd, 1H), 6.55 (d, 1H), 4.56 (s, 2H), 3.95 (t, 2H), 3.19 (t, 2H), 2.88 (t, 2H), 2.54 (t, 2H), 2.48 (bm, 4H), 1.99 (m, 2H), 1.64 (m, 4H), 1.47 (m, 2H); MS (APCI), M+H: 401 (100%). 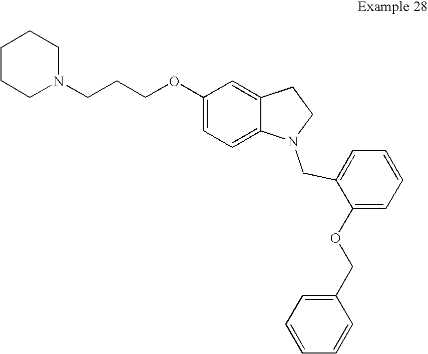
1-(2-Benzyloxy-benzyl)-5-(3-piperidin-1-yl-propoxy)-2,3-dihydro-1H-indole; prepared from 5-(3-piperidin-1-yl-propoxy)-2,3-dihydro-1H-indole dihydrochloride (30 mg, 0.09 mmol) and 2-benzyloxybenzaldehyde (28 μL, 0.18 mmol) by the method of Procedure J (See herein Example 23). The crude material was passed through a short plug of silica gel (20:1 DCM/7N NH₃ in methanol) to give the title compound as a brown oil (34 mg, 83%). ¹H NMR (CDCl₃) δ 7.29-7.44 (m, 6H), 7.23 (m, 1H), 6.95 (m, 2H), 6.75 (d, 1H), 6.57 (dd, 1H), 6.37 (d, 1H), 5.11 (s, 2H), 4.25 (s, 2H), 3.92 (t, 2H), 3.33 (t, 2H), 2.93 (t, 2H), 2.49 (t, 2H), 2.42 (bm, 4H), 1.95 (m, 2H), 1.61 (m, 4H), 1.45 (m, 2H); MS (APCI), M+H: 457 (100%).

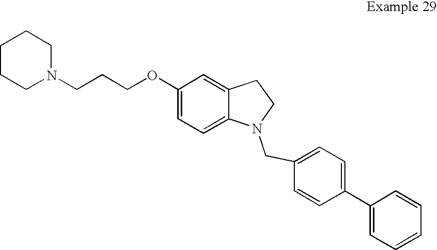

1-Biphenyl-4-ylmethyl-5-(3-piperidin-1-yl-propoxy)-2,3-dihydro-1H-indole; prepared from 5-(3-piperidin-1-yl-propoxy)-2,3-dihydro-1H-indole dihydrochloride (30 mg, 0.09 mmol) and 4-biphenylcarboxaldehyde (33 mg, 0.18 mmol) by the method of Procedure J (See herein Example 23). The crude material was passed through a short plug of silica gel (20:1 DCM/7N NH₃ in methanol) to give the title compound as a waxy, tan solid (31 mg, 81%). ¹H NMR (CDCl₃) S7.55-7.61 (m, 4H), 7.41-7.55 (m, 4H), 7.34 (m, 1H), 6.76 (d, 1H), 6.62 (dd, 1H), 6.44 (d, 1H), 4.20 (s, 2H), 3.93 (t, 2H), 3.27 (t, 2H), 2.93 (t, 2H), 2.52 (t, 2H), 2.46 (bm, 4H), 1.98 (m, 2H), 1.63 (m, 4H), 1.46 (m, 2H); MS (APCI), M+H: 427 (100%). 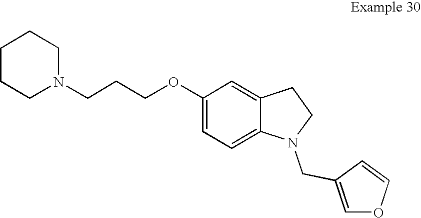
1-Furan-3-ylmethyl-5-(3-piperidin-1-yl-propoxy)-2,3-dihydro-1H-indole; prepared from 5-(3-piperidin-1-yl-propoxy)-2,3-dihydro-1H-indole dihydrochloride (30 mg, 0.09 mmol) and 3-furaldehyde (16 μL, 0.185 mmol) by the method of Procedure J (See herein Example 23). The crude material was passed through a short plug of silica gel (20:1 DCM/7N NH₃ in methanol) to give the title compound as a brown oil (23 mg, 75%). ¹H NMR (CDCl₃) δ 7.37 (m, 2H), 6.74 (dd, 1H), 6.63 (dd, 1H), 6.49 (d, 1H), 6.39 (m, 1H), 4.03 (s, 2H), 3.93 (t, 2H), 3.21 (t, 2H), 2.89 (t, 2H), 2.52 (t, 2H), 2.46 (bm, 4H), 1.98 (m, 2H), 1.63 (m, 4H), 1.46 (m, 2H); MS (APCI), M+H: 341 (100%).

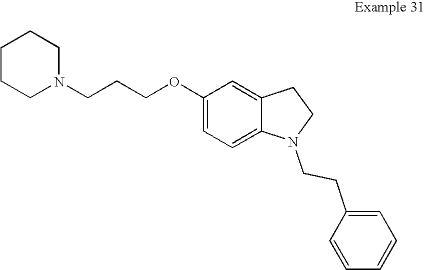

### 1-Phenethyl-5-(3-piperidin-1-yl-propoxy)-2,3-dihydro-1H-indole

Procedure K: (for use with enolizable aldehydes): To a stirred solution of 5-(3-piperidin-1-yl-propoxy)-2,3-dihydro-1H-indole dihydrochloride (25 mg, 0.075 mmol) in 5:1 DCE/methanol (1.2 mL) containing acetic acid (0.1 equivalent) and sodium triacetoxyborohydride (60 mg, 0.0.28 mmol) at room temperature under N₂, was added phenylacetaldehyde (20 μl, 0.154 mmol). After 30 minutes (or until starting material was consumed by TLC) the mixture was loaded directly onto a Varian SCX column (10 g). The column was washed with DCM and methanol, and the desired compound was eluted with a 7N NH₃ in methanol and concentrated in vacuo. Passage of the material through a short plug of silica gel (20:1 solution of DCM/7N NH₃ in methanol) gave 1-phenethyl-5-(3-piperidin-1-yl-propoxy)-2,3-dihydro-1H-indole as a pale yellow, waxy solid (22 mg, 80%). ¹H NMR (CDCl₃) δ 7.20-7.33 (m, 5H), 6.73 (d, 1H), 6.62 (dd, 1H), 6.40 (d, 1H), 3.94 (t, 2H), 3.34 (t, 2H), 3.25 (m, 2H), 2.90 (m, 4H), 2.63 (bm, 6H), 2.08 (bm, 2H), 1.74 (bs, 4H), 1.51 (bs, 2H); MS (ESI), M+H: 365 (100%). 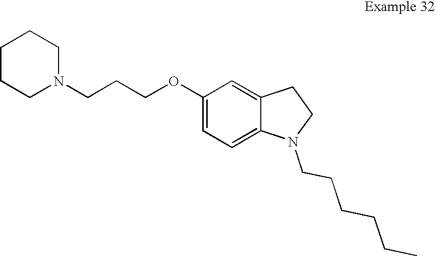
1-Hexyl-5-(3-piperidin-1-yl-propoxy)-2,3-dihydro-1H-indole; prepared as an oil (13 mg, 50%) from 5-(3-piperidin-1-yl-propoxy)-2,3-dihydro-1H-indole dihydrochloride (25 mg, 0.075 mmol) and hexanal (20 μL, 0.167 mmol) by the method of Procedure K (See herein Example 31). ¹H NMR (CDCl₃) δ 6.72 (d, 1H), 6.61 (dd, 1H), 6.38 (d, 1H), 3.93 (t, 2H), 3.25 (t, 2H), 2.95 (t, 1H), 2.90 (t, 1H), 2.61 (bm, 6H), 2.06 (bm, 2H), 1.72 (bm, 4H), 1.58 (m, 2H), 1.50 (bs, 2H), 1.30-1.40 (m, 6H), 0.90 (t, 3H); MS (ESI), M+H: 345 (100%). 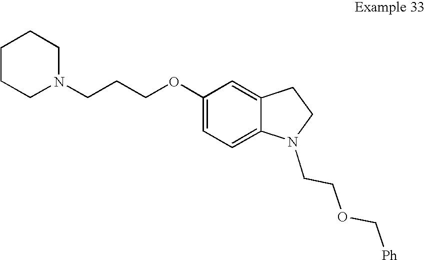
1-(2-Benzyloxy-ethyl)-5-(3-piperidin-1-yl-propoxy)-2,3-dihydro-1H-indole; prepared as a tan, waxy solid (8 mg, 27%) from 5-(3-piperidin-1-yl-propoxy)-2,3-dihydro-1H-indole dihydrochloride (25 mg, 0.075 mmol) and benzyloxyacetaldehyde (20 μL, 0.14 mmol) by the method of Procedure K (See herein Example 31). ¹H NMR (CDCl₃) δ 7.27-7.36 (m, 5H), 6.69 (d, 1H), 6.58 (dd, 1H), 6.40 (d, 1H), 4.57 (s, 2H), 3.95 (t, 2H), 3.69 (t, 2H), 3.36 (t, 2H), 3.23 (t, 2H), 2.98 (bs, 6H), 2.92 (t, 2H), 2.27 (bm, 2H), 1.93 (bm, 4H), 1.59 (bs, 2H); MS (ESI), M+H: 395 (100%). 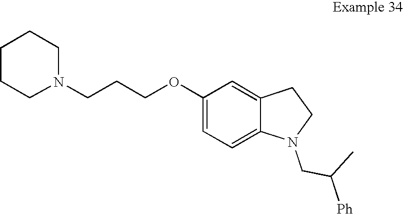
1-(2-Phenyl-propyl)-5-(3-piperidin-1-yl-propoxy)-2,3-dihydro-1H-indole; prepared as an oil (32 mg, 94%) from 5-(3-piperidin-1-yl-propoxy)-2,3-dihydro-1H-indole dihydrochloride (25 mg, 0.075 mmol) and 2-phenylpropionaldehyde (20 μL, 0.15 mmol) by the method of Procedure K (See herein Example 31). ¹H NMR (CDCl₃) δ7.25-7.33 (m, 4H), 7.21 (m, 1H), 6.70 (bs, 1H), 6.59 (bd, 1H), 6.33 (d, 1H), 3.91 (t, 2H), 3.03-3.29 (m, 5H), 2.86 (t, 2H), 2.54 (bm, 6H), 1.99 (bm, 2H), 1.65 (bm, 4H), 1.46 (bs, 2H), 1.34 (d, 3H); MS (ESI), M+H: 379 (100%). 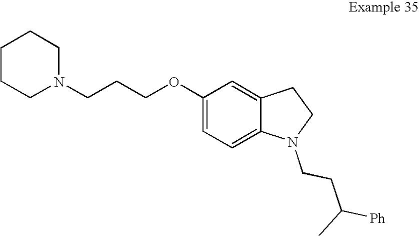
1-(3-Phenyl-butyl)-5-(3-piperidin-1-yl-propoxy)-2,3-dihydro-1H-indole; prepared as an oil (30 mg, 85%) from 5-(3-piperidin-1-yl-propoxy)-2,3-dihydro-1H-indole dihydrochloride (25 mg, 0.075 mmol) and 3-phenylbutyraldehyde (20 μL, 0.135 mmol) by the method of Procedure K (See herein Example 31). ¹H NMR (CDCl₃) δ 7.28-7.33 (m, 2H), 7.18-7.23 (m, 3H), 6.71 (d, 1H), 6.57 (dd, 1H), 6.22 (d, 1H), 3.91 (t, 2H), 3.26 (q, 1H), 3.18(q, 1H), 2.78-2.94 (m, 5H), 2.54 (t, 2H), 2.49 (bm, 4H), 1.98 (m, 2H), 1.86 (m, 2H), 1.65 (m, 4H), 1.46 (bm, 2H), 1.32 (d, 3H); MS (ESI), M+H: 393 (100%). 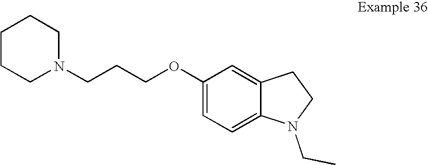
1-Ethyl-5-(3-piperidin-1-yl-propoxy)-2,3-dihydro-1H-indole; prepared as a white solid (20 mg, 92%) from 5-(3-piperidin-1-yl-propoxy)-2,3-dihydro-1H-indole dihydrochloride (25 mg, 0.075 mmol) and acetaldehyde (excess) by the method of Procedure K (See herein Example 31). ¹H NMR (CDCl₃) δ 6.72 (d, 1H), 6.61 (dd, 1H), 6.41 (d, 1H), 3.94 (t, 2H), 3.25 (t, 2H), 3.05 (q, 2H), 2.90 (t, 1H), 2.67 (t, 2H), 2.62 (bs, 4H), 2.08 (bm, 2H), 1.75 (bm, 4H), 1.51 (m, 2H), 1.18 (t, 3H); MS (ESI), M+H: 289 (100%). 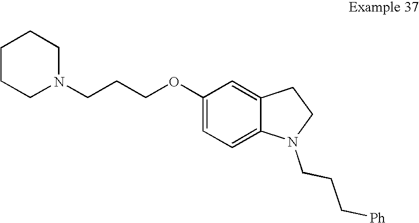
1-(3-Phenyl-propyl)-5-(3-piperidin-1-yl-propoxy)-2,3-dihydro-1H-indole; prepared as pale yellow, waxy solid (26 mg, 90%) from 5-(3-piperidin-1-yl-propoxy)-2,3-dihydro-1H-indole dihydrochloride (25 mg, 0.075 mmol) and hydrocinnamaldehyde (25 μL, 0.17 mmol) by the method of Procedure K (See herein Example 31). ¹H NMR (CDCl₃) δ 7.29 (m, 2H), 7.21 (m, 3H), 6.73 (d, 1H), 6.60 (dd, 1H), 6.33 (d, 1H), 3.93 (t, 2H), 3.27 (t, 2H), 2.99 (t, 2H), 2.91 (t, 2H), 2.73 (t, 2H), 2.58 (bm, 6H), 2.03 (bm, 2H), 1.92 (m, 2H), 1.70 (bm, 4H), 1.49 (bs, 2H); MS (ESI), M+H: 379 (100%). 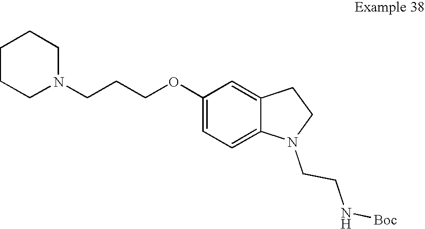
{2-[5-(3-Piperidin-1-yl-propoxy)-2,3-dihydro-indol-1-yl]-ethyl}-carbamic acid tert-butyl ester; prepared as a pale oil (18 mg, 20%) from 5-(3-piperidin-1-yl-propoxy)-2,3-dihydro-1H-indole dihydrochloride (75 mg, 0.225 mmol) and (2-oxoethyl)-carbamic acid tert-butyl ester (55 mg, 0.346 mmol) by the method of Procedure K (See herein Example 31), except that the crude reaction mixture was treated with PS-hydrazide (Argonaut) and triamine-3 (Silicycle) and filtered prior to chromatography. ¹H NMR (CDCl₃) δ 6.74 (s, 1H), 6.63 (d, 1H), 6.42 (d, 1H), 4.91 (bs, 1H), 3.93 (t, 2H), 3.36 (bm, 2H), 3.28 (t, 2H), 3.10 (t, 2H), 2.93 (t, 2H), 2.52 (bm, 6H), 1.99 (bm, 2H), 1.65 (bm, 4H), 1.45 (bs, 11H); MS (ESI), M+H: 404 (60%), M+H-Boc: 304 (100%).

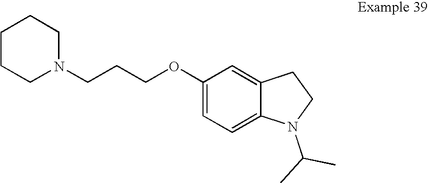

### 1-Isopropyl-5-(3-piperidin-1-yl-propoxy)-2,3-dihydro-1H-indole

Procedure L:

A stirred solution of 5-(3-piperidin-1-yl-propoxy)-2,3-dihydro-1H-indole dihydrochloride (30 mg, 0.09 mmol), acetone (1 mL), and sodium cyanoborohydride (18 mg, 0.283 mmol) in 1:1 DCE/methanol containing acetic acid (3 drops) was heated to 50° C. in a sealed tube overnight. After cooling to room temperature, the mixture was loaded directly onto a Varian SCX column (10 g). The column was washed with DCM and methanol, and the desired compound was then eluted with 7N NH₃ in methanol and is isolated as a brown oil (16 mg, 59%). ¹H NMR (CDCl₃) δ 6.72 (m, 1H), 6.62 (dd, 1H), 6.33 (d, 1H), 3.91 (t, 2H), 3.74 (sept, 1H), 3.26 (t, 2H), 2.88 (t, 2H), 2.46 (t, 2H), 2.39 (bm, 4H), 1.93 (m, 2H), 1.59 (m, 4H), 1.44 (m, 2H) 1.12 (d, 6H); MS (APCI), M+H: 303 (100%).

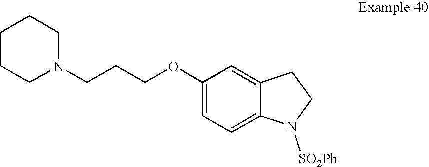

### 1-Benzenesulfonyl-5-(3-piperidin-1-yl-propoxy)-2,3-dihydro-1H-indole

Procedure M: To a stirred mixture of 5-(3-piperidin-1-yl-propoxy)-2,3-dihydro-1H-indole dihydrochloride (40 mg, 0.12 mmol) and triethylamine (0.2 mL) at room temperature in dry 10:1:1 tetrahydrofuran (THF)/DCM/DMF (12 mL) under dry N₂ was added pyridine-2 (Silicycle, 1.76 mmol/g, 400 mg, 0.70 mmol). After 10 minutes, benzenesulfonyl chloride (19 μL, 0.148 mmol) was added and stirring was continued for 4 hours (or until starting material was consumed by TLC). Triamine-3 (Silicycle, 1.42 mmol/g, 250 mg, 0.355 mmol) was added, and after 30 minutes the mixture was suction filtered, the scavenger was rinsed with DCM, and the combined filtrates were concentrated in vacuo. Flash chromatography on silica gel or preparative TLC (20:1 DCM/7N NH₃ in methanol) furnished the title compound (22 mg, 45%) as a pale yellow oil. ¹H NMR (CDCl₃) δ 7.72 (d, 2H), 7.54 (t, 2H), 7.42 (t, 2H), 6.74 (dd, 1H), 6.62 (d, 1H), 3.93 (t, 2H), 3.91 (t, 2H), 2.72 (t, 2H), 2.45 (t, 2H), 2.40 (bs, 4H), 1.94 (m, 2H), 1.59 (m, 4H), 1.45 (bm, 2H); MS (ESI), M+H: 401 (100%). 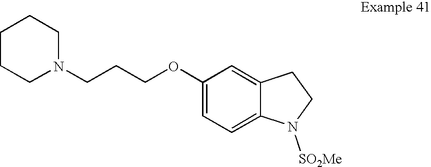
1-Methanesulfonyl-5-(3-piperidin-1-yl-propoxy)-2,3-dihydro-1H-indole; prepared as a waxy, white solid (9 mg, 22%) from 5-(3-piperidin-1-yl-propoxy)-2,3-dihydro-1H-indole dihydrochloride (40 mg, 0.12 mmol) and methanesulfonyl chloride (12 μL, 0.155 mmol) by the method of Procedure M (See herein Example 40). ¹H NMR (CDCl₃) δ 7.31 (d, 1H), 6.79 (d, 1H), 6.73 (dd, 1H), 3.97 (m, 4H), 3.11 (t, 2H), 2.81 (s, 3H), 2.49 (t, 2H), 2.43 (bs, 4H), 1.98 (m, 2H), 1.62 (m, 4H), 1.46 (bm, 2H); MS (ESI), M+H: 339 (100%). 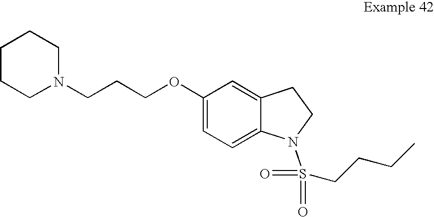
1-Butanesulfonyl-5-(3-piperidin-1-yl-propoxy)-2,3-dihydro-1H-indole; prepared as a pale oil (18 mg, 40%) from 5-(3-piperidin-1-yl-propoxy)-2,3-dihydro-1H-indole dihydrochloride (40 mg, 0.12 mmol) and 1-butanesulfonyl chloride (19 μL, 0.147 mmol) by the method of Procedure M (See herein Example 40). ¹H NMR (CDCl₃) δ 7.26 (d, 1H), 6.78 (d, 1H), 6.70 (dd, 1H), 4.01 (t, 2H), 3.96 (t, 2H), 3.10 (t, 2H), 2.96 (m, 2H), 2.49 (t, 2H), 2.42 (bs, 4H), 1.97 (m, 2H), 1.80 (m, 2H), 1.61 (m, 4H), 1.45 (bm, 2H), 1.41 (m, 2H), 0.91 (t, 3H); MS (ESI), M+H: 381 (100%). 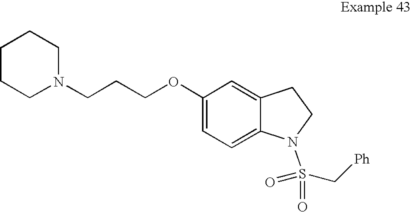
1-Phenylmethanesulfonyl-5-(3-piperidin-1-yl-propoxy)-2,3-dihydro-1H-indole; prepared as a waxy, white solid (14 mg, 28%) from 5-(3-piperidin-1-yl-propoxy)-2,3-dihydro-1H-indole dihydrochloride (40 mg, 0.12 mmol) and α-toluenesulfonyl chloride (28 mg, 0.147 mmol) by the method of Procedure M (See herein Example 40). ¹H NMR (CDCl₃) δ 7.24-7.34 (m, 4H), 7.20 (m, 2H), 6.74 (m, 1H), 6.71 (m, 1H), 4.32 (s, 2H), 3.99 (t, 2H), 3.64 (t, 2H), 2.80 (t, 2H), 2.53 (bm, 6H), 2.03 (bm, 2H), 1.66 (bm, 4H), 1.48 (bm, 2H); MS (ESI), M+H: 415 (100%).

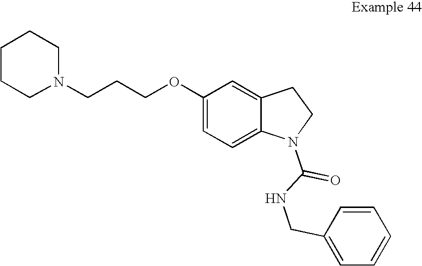

### 5-(3-Piperidin-1-yl-propoxy)-2,3-dihydro-indole-1-carboxylic acid benzylamide

Procedure N: To a stirred mixture of 5-(3-piperidin-1-yl-propoxy)-2,3-dihydro-1H-indole dihydrochloride (40 mg, 0.12 mmol) and triethylamine (0.2 mL) at room temperature in dry 10:1:1 THF/DCM/DMF (12 mL) under dry N₂ was added pyridine-2 (Silicycle, 1.76 mmol/g, 400 mg, 0.70 mmol). After 10 minutes, benzyl isocyanate (18 μL, 0.146 mmol) was added and stirring was continued for 4 hours (or until starting material was consumed by TLC). Triamine-3 (Silicycle, 1.42 mmol/g, 250 mg, 0.355 mmol) was added, and after 30 minutes the mixture was suction filtered, the scavenger was rinsed with DCM, and the combined filtrates were concentrated in vacuo. Flash chromatography on silica gel or preparative TLC (20:1 DCM/7N NH₃ in methanol) furnished the title compound (33 mg, 70%) as a brown oil. ¹H NMR (CDCl₃) δ 7.79 (d, 1H), 7.25-7.37 (m, 5H), 6.73 (m, 1H), 6.70 (dd, 1H), 4.81 (t, 1H), 4.52 (d, 2H), 3.95 (t, 2H), 3.90 (t, 2H), 3.13 (t, 2H), 2.48 (t, 2H), 2.42 (bm, 4H), 1.96 (m, 2H), 1.61 (m, 4H), 1.45 (m, 2H); MS (APCI), M+H: 394 (100%). 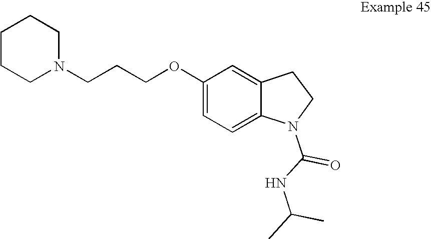
5-(3-Piperidin-1-yl-propoxy)-2,3-dihydro-indole-1-carboxylic acid isopropylamide; prepared as a waxy, tan solid (30 mg, 72%) from 5-(3-piperidin-1-yl-propoxy)-2,3-dihydro-1H-indole dihydrochloride (40 mg, 0.12 mmol) and isopropyl isocyanate (15 μL, 0.152 mmol) by the method of Procedure N (See herein Example 44). ¹H NMR (CDCl₃) δ 7.74 (d, 1H), 6.72 (m, 1H), 6.89 (dd, 1H), 4.28 (bd, 1H), 4.06 (sept, 1H), 3.95 (t, 2H), 3.86 (t, 2H), 3.12 (t, 2H), 2.48 (t, 2H), 2.42 (bm, 4H), 1.95 (m, 2H), 1.60 (m, 4H), 1.45 (m, 2H); MS (APCI), M+H: 346 (100%). 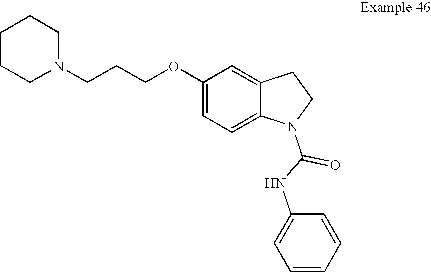
5-(3-Piperidin-1-yl-propoxy)-2,3-dihydro-indole-1-carboxylic acid phenylamide; prepared as a brown oil (24 mg, 53%) from 5-(3-piperidin-1-yl-propoxy)-2,3-dihydro-1H-indole dihydrochloride (40 mg, 0.12 mmol) and phenyl isocyanate (16 μL, 0.147 mmol) by the method of Procedure N (See herein Example 44). ¹H NMR (CDCl₃) δ 7.78 (d, 1H), 7.44 (m, 2H), 7.31 (m, 2H), 7.06 (m, 1H), 6.76 (m, 1H), 6.72 (dd, 1H), 6.44 (bs, 1H), 4.06 (t, 2H), 3.96 (t, 2H), 3.18 (t, 2H), 2.48 (t, 2H), 2.42 (bm, 4H), 1.96 (m, 2H), 1.60 (m, 4H), 1.45 (m, 2H); MS (APCI), M+H: 380 (100%). 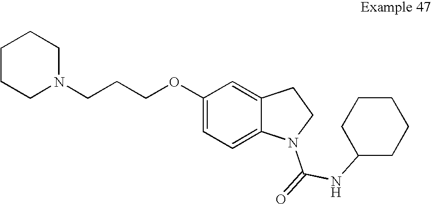
5-(3-Piperidin-1-yl-propoxy)-2,3-dihydro-indole-1-carboxylic acid cyclohexylamide; prepared as an off-white solid (26 mg, 56%) from 5-(3-piperidin-1-yl-propoxy)-2,3-dihydro-1H-indole dihydrochloride (40 mg, 0.12 mmol) and cyclohexyl isocyanate (19 μL, 0.149 mmol) by the method of Procedure N (See herein Example 44). ¹H NMR (CDCl₃) δ 7.75 (d, 1H), 6.70 (bs, 1H), 6.67 (bd, 1H), 4.33 (bd, 1H), 3.96 (t, 2H), 3.86 (t, 2H), 3.74 (m, 1H), 3.12 (t, 2H), 2.61 (bm, 6H), 2.02 (m, 2H), 1.59-1.75 (bm, 8H), 1.50 (bs, 2H), 1.39 (m, 2H), 1.17 (m, 2H); MS (APCI), M+H: 386 (100%). 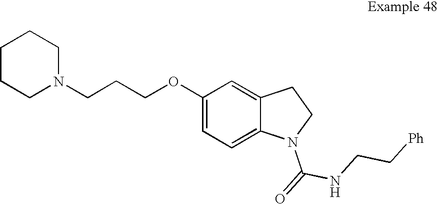
5-(3-Piperidin-1-yl-propoxy)-2,3-dihydro-indole-1-carboxylic acid phenethylamide; prepared as a pale oil (28 mg, 57%) from 5-(3-piperidin-1-yl-propoxy)-2,3-dihydro-1H-indole dihydrochloride (40 mg, 0.12 mmol) and phenethyl isocyanate (20 μL, 0.145 mmol) by the method of Procedure N (See herein Example 44). ¹H NMR (CDCl₃) δ 7.70 (d, 1H), 7.32 (m, 2), 7.24 (m, 3H), 6.71 (d, 1H), 6.67 (dd, 1H), 4.50 (bt, 1H), 3.95 (t, 2H), 3.76 (t, 2H), 3.58 (q, 2H), 3.09 (t, 2H), 2.88 (t, 2H), 2.49 (t, 2H), 2.43 (bs, 4H), 1.96 (m, 2H), 1.61 (m, 2H), 1.45 (m, 2H); MS (APCI), M+H: 408 (100%).

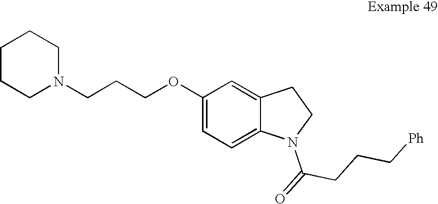

### 4-Phenyl-1-[5-(3-piperidin-1-yl-propoxy)-2,3-dihydro-indol-1-yl]-butan-1-one;

Procedure P: A mixture of 5-(3-piperidin-1-yl-propoxy)-2,3-dihydro-1H-indole dihydrochloride (100 mg, 0.3 mmol), 4-phenylbutyric acid (100 mg, 0.61 mmol), N-cyclohexylcarbodiimide-N′-methyl polystyrene HL (Novabiochem, 1.7 mmol/g, 600 mg, 1.02 mmol), triethylamine (0.125 mL, 0.9 mmol) and HOBt (60 mg, 0.45 mmol) in dry 5:1:1 chloroform/acetonitrile/tert-butanol (10 mL) under dry N₂ was stirred at room temperature overnight. Triamine-3 (Silicycle, 1.42 mmol/g, 800 mg, 1.14 mmol) was added and stirring was continued for 30-60 minutes. The mixture was suction filtered, the scavenger was rinsed with DCM, and the combined filtrates were concentrated in vacuo. The residue was loaded onto a Varian SCX column (10 g), the column was washed with DCM and methanol, and eluted with 7N NH₃ in methanol, to give 4-phenyl-1-[5-(3-piperidin-1-yl-propoxy)-2,3-dihydro-indol-1-yl]-butan-1-one quantitatively (˜150 mg) as a single peak in the LCMS. A portion of the material was further purified by flash chromatography on silica gel (20:1 DCM/7N NH₃ in methanol) to provide the title compound as a white solid (20 mg). ¹H NMR (CDCl₃) δ 8.13 (d, 1H), 7.29 (m, 2H), 7.21 (m, 3H), 6.69 (bs, 1H), 6.67 (dd, 1H), 3.96 (t, 2H), 3.94 (t, 2H), 3.11 (t, 2H), 2.78 (bm, 6H), 2.74 (t, 2H), 2.39 (t, 2H), 2.14 (bs, 2H), 2.08 (qt, 2H), 1.79 (bs, 4H), 1.54 (bs, 2H); MS (ESI), M+H: 407 (100%). 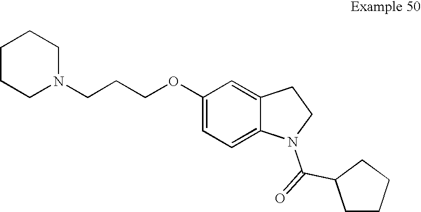
4-Cyclopentyl-[5-(3-piperidin-1-yl-propoxy)-2,3-dihydro-indol-1-yl]-methanone; prepared as a waxy, white solid (95 mg, 88%) from 5-(3-piperidin-1-yl-propoxy)-2,3-dihydro-1H-indole dihydrochloride (100 mg, 0.3 mmol) and cyclopentanecarboxylic acid (70 μL, 0.61 mmol), by the method of Procedure P (See herein Example 49). ¹H NMR (CDCl₃) δ 8.15 (d, 1H), 6.73 (bs, 1H), 6.69 (dd, 1H), 4.11 (t, 2H), 3.96 (t, 2H), 3.15 (t, 2H), 2.90 (qt, 1H), 2.57 (t, 2H), 2.52 (bs, 4H), 2.00 (m, 2H), 1.90 (m, 4H), 1.80 (m, 2H), 1.64 (m, 6H), 1.47 (bm, 2H); MS (APCI), M+H: 357 (100%). 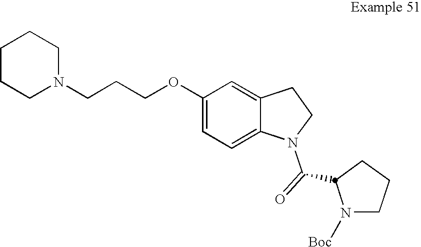
(S)-2-[5-(3-Piperidin-1-yl-propoxy)-2,3-dihydro-indole-1-carbonyl]-pyrrolidine-1-carboxylic acid tert-butyl ester; prepared in quantitative yield (−200 mg) as a single peak in the LCMS from 5-(3-piperidin-1-yl-propoxy)-2,3-dihydro-1H-indole dihydrochloride (150 mg, 0.45 mmol) and N-(tert-butoxycarbonyl)-L-proline (193 mg, 0.90 mmol) by the method of Procedure P (See herein Example 49). A portion of the material was further purified by flash chromatography on silica gel (20:1 DCM/7N NH₃ in methanol) to provide the title compound as a waxy, white solid (33 mg). ¹H NMR (CDCl₃) δ 8.15 (d, 0.5H), 8.12 (d, 0.5H), 6.67-6.76 (m, 2H), 4.60 (bdd, 0.5H), 4.44 (dd, 0.5H), 4.26-4.41 (m, 1H), 4.00-4.08 (m, 1H), 3.98 (t, 1H), 3.96 (t, 1H), 3.66 (m, 1H), 3.49 (m, 1H), 3.17 (m, 2H), 2.51 (t, 2H), 2.45 (bs, 4H), 1.98 (m, 2H), 1.84-2.28 (m, 4H), 1.62 (m, 4H), 1.46 (s, 4.5H), 1.42 (bm, 2H), 1.34 (s, 4.5H); MS (APCI), M+H: 458 (100%), M+H-Boc: 358 (40%). 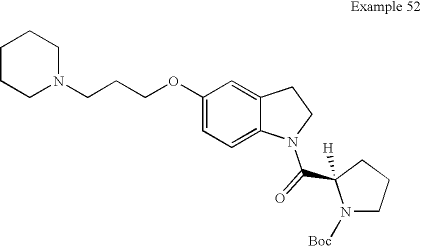
(R)-2-[5-(3-Piperidin-1-yl-propoxy)-2,3-dihydro-indole-1-carbonyl]-pyrrolidine-1-carboxylic acid tert-butyl ester; prepared in quantitative yield (−400 mg) as a single peak in the LCMS from 5-(3-piperidin-1-yl-propoxy)-2,3-dihydro-1H-indole dihydrochloride (300 mg, 0.9 mmol) and N-(tert-butoxycarbonyl)-D-proline (390 mg, 1.81 mmol) by the method of Procedure P (See herein Example 49). A portion of the material was further purified by flash chromatography on silica gel (20:1 DCM/7N NH₃ in methanol) to provide the title compound as a yellow oil (45 mg). ¹H NMR (CDCl₃) δ 8.16 (d, 0.5H), 8.12 (d, 0.5H), 6.68-6.76 (m, 2H), 4.60 (bdd, 0.5H), 4.44 (dd, 0.5H), 4.39 (m, 0.5H), 4.18 (m, 0.5H), 4.00-4.08 (m, 1H), 3.98 (t, 1H), 3.96 (t, 1H), 3.66 (m, 1H), 3.49 (m, 1H), 3.18 (m, 2H), 2.48 (t, 2H), 2.42 (bs, 4H), 1.98 (m, 2H), 1.84-2.28 (m, 4H), 1.61 (m, 4H), 1.46 (s, 4.5H), 1.42 (bm, 2H), 1.34 (s, 4.5H); MS (APCI), M+H: 458 (100%), M+H-Boc: 358 (40%). 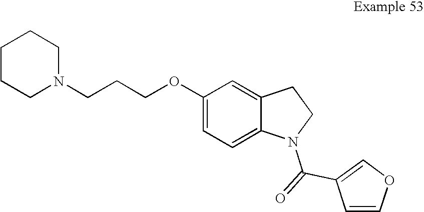
Furan-3-yl-[5-(3-piperidin-1-yl-propoxy)-2,3-dihydro-indol-1-yl]-methanone; prepared as a white solid (31 mg, 61%) from 5-(3-piperidin-1-yl-propoxy)-2,3-dihydro-1H-indole dihydrochloride (48 mg, 0.144 mmol) and 3-furoic acid (21 mg, 0.187 mmol) by the method of Procedure P (See herein Example 49). ¹H NMR (CDCl₃) δ 8.15 (bs, 1H), 7.86 (s, 1H), 7.46 (d, 1H), 6.73-6.78 (bm, 3H), 4.23 (t, 2H), 3.98 (t, 2H), 3.16 (t, 2H), 2.47 (t, 2H), 2.41 (bs, 4H), 1.97 (m, 2H), 1.60 (m, 4H), 1.45 (bm, 2H); MS (APCI), M+H: 355 (100%). 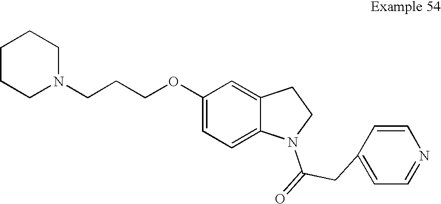
1-[5-(3-Piperidin-1-yl-propoxy)-2,3-dihydro-indol-1-yl]-2-pyridin-3-yl-ethanone; prepared as an off-white solid (53 mg, 56%) from 5-(3-piperidin-1-yl-propoxy)-2,3-dihydro-1H-indole dihydrochloride (70 mg, 0.21 mmol) and 3-pyridylacetic acid (48 mg, 0.35 mmol) by the method of Procedure P (See herein Example 49). ¹H NMR (CDCl₃) δ 8.53 (s, 2H), 8.12 (d, 1H), 7.71 (d, 1H), 7.28 (dd, 1H), 6.75 (s, 1H), 6.71 d, 1H), 4.10 (t, 2H), 3.96 (t, 2H), 3.77 (s, 2H), 3.17 (t, 2H), 2.46 (t, 2H), 2.40 (bs, 4H), 1.95 (m, 2H), 1.60 (m, 4H), 1.44 (bm, 2H); MS (APCI), M+H: 380 (100%). 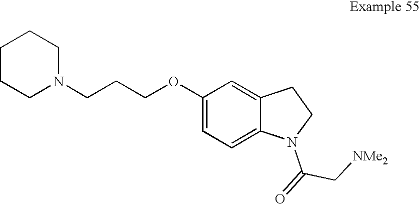
2-Dimethylamino-1-[5-(3-piperidin-1-yl-propoxy)-2,3-dihydro-indol-1-yl]-ethanone; prepared as a pale oil (100 mg, 60%) from 5-(3-piperidin-1-yl-propoxy)-2,3-dihydro-1H-indole dihydrochloride (140 mg, 0.42 mmol) and N,N-dimethyl glycine (100 mg, 0.97 mmol) by the method of Procedure P (See herein Example 49). ¹H NMR (CDCl₃) δ 8.14 (d, 1H), 6.74 (bs, 1H), 6.71 (dd, 1H), 4.15 (t, 2H), 3.98 (t, 2H), 3.17 (s, 2H), 3.14 (t, 2H), 2.55 (bt, 2H), 2.49 (bs, 4H), 2.36 (s, 6H), 2.01 (bm, 2H), 1.65 (bm, 4H), 1.46 (bm, 1H); MS (ESI), M+H: 346 (100%). 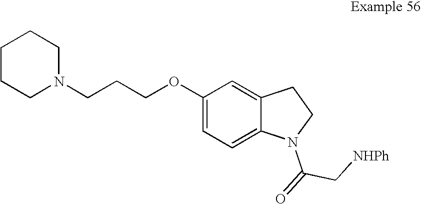
2-Phenylamino-1-[5-(3-piperidin-1-yl-propoxy)-2,3-dihydro-indol-1-yl]-ethanone; prepared as a pale oil (100 mg, 60%) from 5-(3-piperidin-1-yl-propoxy)-2,3-dihydro-1H-indole dihydrochloride (140 mg, 0.42 mmol) and N-phenyl glycine (190 mg, 1.26 mmol) by the method of Procedure P (See herein Example 49). ¹H NMR (CDCl₃) δ 8.13 (d, 1H), 7.21 (t, 2H), 6.75 (m, 3H), 6.67 (d, 2H), 4.91 (bt, 1H), 4.07 (t, 2H), 3.99 (t, 2H), 3.94 (d, 2H), 3.23 (t, 2H), 2.57 (bt, 2H), 2.51 (bs, 4H), 2.03 (bm, 2H), 1.67 (bm, 4H), 1.47 (bm, 11H); MS (ESI), M+H: 394 (100%). 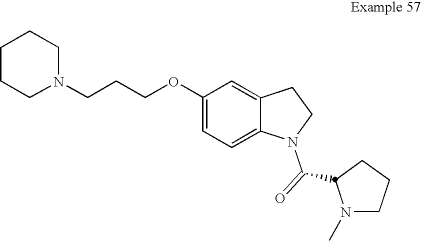
(S)-(1-Methyl-pyrrolidin-2-yl)-[5-(3-piperidin-1-yl-propoxy)-2,3-dihydro-indol-1-yl]-methanone; prepared as a tan solid (120 mg, 77%) from 5-(3-piperidin-1-yl-propoxy)-2,3-dihydro-1H-indole dihydrochloride (140 mg, 0.42 mmol) and N-methyl-L-proline (125 mg, 0.97 mmol) by the method of Procedure P (See herein Example 49). ¹H NMR (CDCl₃) δ 8.20 (d, 1H), 6.75 (bs, 1H), 6.72 (dd, 1H), 4.14 (m, 2H), 3.97 (t, 2H), 3.10-3.22 (m, 4H), 2.47 (bt, 2H), 2.41 (bs, 7H), 2.34 (m, 1H), 2.18 (m, 1H), 1.92-2.04 (m, 4H), 1.82 (bm, 1H), 1.59 (m, 4H), 1.44 (bm, 2H); MS (ESI), M+H: 372 (100%).

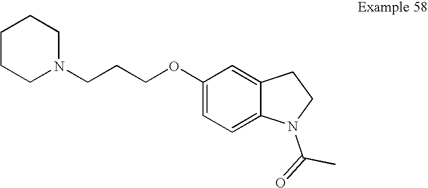

### 1-[5-(3-Piperidin-1-yl-propoxy)-2,3-dihydro-indol-1-yl]-ethanone

Procedure O: To a stirred mixture of 5-(3-piperidin-1-yl-propoxy)-2,3-dihydro-1H-indole dihydrochloride (35 mg, 0.105 mmol), PS-DMAP (Argonaut, 1.48 mmol/g, 14 mg, 0.02 mmol), PS-DIEA (Argonaut, 3.83 mmol/g, 120 mg, 0.46 mmol) and triethylamine (2 μL, 0.014 mmol) in dry DCM (3 mL) at room temperature under dry N₂ was added acetyl chloride (22 μL, 0.309 mmol). After 2 hours, triamine-3 (Silicycle, 1.42 mmol/g, 300 mg, 0.426 mmol) and isocyanate-3 (Silicycle, 1.21 mmol/g, 350 mg, 0.42 mmol) were added and stirring was continued for several hours. The mixture was suction filtered, the scavengers were rinsed with DCM, and the filtrate was concentrated in vacuo. Purification of the residue by flash chromatography on silica (20:1 DCM/7N NH₃ in methanol), gave the title compound as a waxy, tan solid (26 mg, 82%). ¹H NMR (CDCl₃) δ 8.10 (d, 1H), 6.74 (d, 1H), 6.71 (dd, 1H), 4.03 (t, 2H), 3.97 (t, 2H), 3.15 (t, 2H), 2.49 (t, 2H), 2.43 (bs, 4H), 2.20 (s, 3H), 1.97 (m, 2H), 1.61 (m, 4H), 1.45 (bm, 2H); MS (ESI), M+H: 303 (60%). 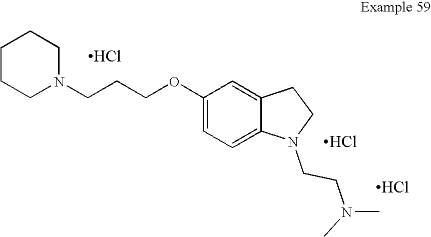
Dimethyl-{2-[5-(3-piperidin-1-yl-propoxy)-2,3-dihydro-indol-1-yl]-ethyl}-amine, trihydrochloride; prepared from 2-dimethylamino-1-[5-(3-piperidin-1-yl-propoxy)-2,3-dihydro-indol-1-yl]-ethanone (87 mg, 0.252 mmol) by the method of Procedure R (See herein Example 61). The free base was converted to its trihydrochloride with 2M HCl in ether/DCM, and was isolated as a tan solid (53 mg, 48%). ¹H NMR (free base in CDCl₃) δ 6.73 (bs, 1H), 6.62 (dd, 1H), 6.40 (d, 1H), 3.92 (t, 2H), 3.30 (t, 2H), 3.11 (t, 2H), 2.91 (t, 2H), 2.53 (t, 2H), 2.49 (t, 2H), 2.43 (bs, 4H), 2.31 (s, 6H), 1.95 (m, 2H), 1.61 (m, 4H), 1.45 (bm, 2H); MS (APCI), M+H: 332 (100%). 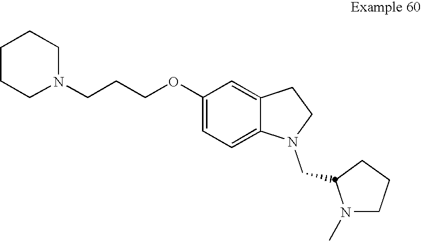
(S)-1-(1-Methyl-pyrrolidin-2-ylmethyl)-5-(3-piperidin-1-yl-propoxy)-2,3-dihydro-1H-indole; prepared from (S)-(1-methyl-pyrrolidin-2-yl)-[5-(3-piperidin-1-yl-propoxy)-2,3-dihydro-indol-1-yl]-methanone (74 mg, 0.199 mmol) by the method of Procedure R (See herein Example 61). The free base was converted to its trihydrochloride with 2M HCl in ether/DCM, and was isolated as a brown solid (62 mg, 67%). ¹H NMR (free base in CDCl₃) δ 6.73 (d, 1H), 6.62 (dd, 1H), 6.40 (d, 1H), 3.91 (t, 2H), 3.32 (m, 2H), 3.07-3.14 (m, 2H), 2.87-2.93 (m, 3H), 2.45 (s, 3H), 2.37-2.47 (m, 7H), 2.22 (m, 1H), 2.02 (m, 1H), 1.92 (m, 2H), 1.62-1.85 (m, 3H), 1.58 (m, 4H), 1.43 (bm, 2H); MS (APCI), M+H: 358 (100%).

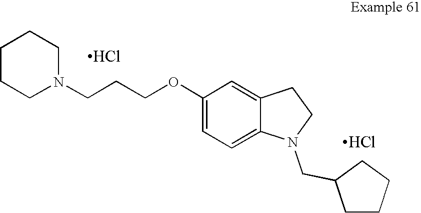

### 1-Cyclopentylmethyl-5-(3-piperidin-1-yl-propoxy)-2,3-dihydro-1H-indole dihydrochloride

Procedure R: A stirred solution of 4-cyclopentyl-[5-(3-piperidin-1-yl-propoxy)-2,3-dihydro-indol-1-yl]-methanone (60 mg, 0.169 mmol) and lithium aluminum hydride (1M in THF, 0.4 mL, 0.4 mmol) in dry THF (5 mL) under dry N₂ was refluxed for 3 hours, cooled to 0° C., and quenched cautiously with excess sodium sulfate decahydrate. After stirring for 1-2 additional hours, the mixture was filtered with suction, the precipitated salts were washed with additional THF, and the combined filtrates concentrated in vacuo. The residue was loaded directly onto a Varian SCX column (10 g). The column was washed with DCM and methanol, and the desired compound was then eluted with 7N NH₃ in methanol. Further purification by flash chromatography on silica gel or preparative TLC (20:1 DCM/7N NH₃ in methanol) afforded the title compound contaminated with some of the analogous indole compound.
Procedure S: The indole was removed by brief exposure to sodium cyanoborohydride in DCM/methanol in the presence of acetic acid. The free base was treated with 2M HCL in ether/DCM to furnish the title compound (46 mg, 66%) as a gray-brown solid. ¹H NMR (free base in CDCl₃) δ 6.72 (d, 1H), 6.61 (dd, 1H), 6.37 (d, 1H), 3.92 (t, 2H), 3.29 (t, 2H), 2.91 (t, 2H), 2.85 (d, 2H), 2.55 (t, 2H), 2.50 (bs, 41), 2.19 (sept, 1H), 1.99 (m, 2H), 1.81 (m, 2H), 1.65 (m, 6H), 1.57 (m, 2H), 1.47 (bm, 2H), 1.28 (m, 2H); MS (APCI), M+H: 343 (100%).

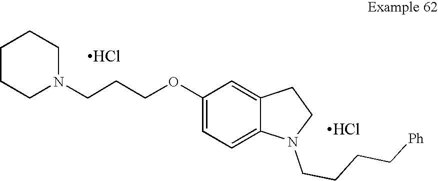

### 1-(4-Phenyl-butyl)-5-(3-piperidin-1-yl-propoxy)-2,3-dihydro-1H-indole dihydrochloride

prepared as a gray-brown solid (15 mg, 11%) from 4-phenyl-1-[5-(3-piperidin-1-yl-propoxy)-2,3-dihydro-indol-1-yl]-butan-1-one; (125 mg, 0.3 mmol) by the method of Procedures R and S (See herein Example 61). ¹H NMR (free base in CDCl₃) δ 7.28 (m, 2H), 7.19 (m, 3H), 6.71 (bs, 1H), 6.60 (dd, 1H), 6.36 (d, 1H), 3.93 (t, 2H), 3.23 (t, 2H), 2.97 (t, 2H), 2.89 (t, 2H), 2.67 (t, 2H), 2.63-2.70 (bm, 6H), 2.09 (bm, 2H), 1.69-1.78 (m, 6H), 1.64 (m, 2H), 1.52 (bs, 2H); MS (ESI), M+H: 393 (100%). 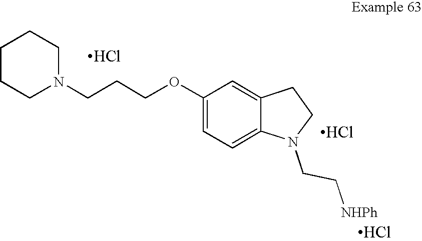
Phenyl-{2-[5-(3-piperidin-1-yl-propoxy)-2,3-dihydro-indol-1-yl]-ethyl}-amine, trihydrochloride; prepared as a tan solid (20 mg, 16%) from 4-phenyl-1-[5-(3-piperidin-1-yl-propoxy)-2,3-dihydro-indol-1-yl]-butan-1-one; (125 mg, 0.3 mmol) by the method of Procedures R and S (See herein Example 61). ¹H NMR (free base in CDCl₃) δ 7.19 (t, 2H), 6.75 (dd, 1H), 6.72 (t, 1H), 6.66 (d, 2H), 6.63 (d, 1H), 6.45 (d, 1H), 4.15 (bs, 1H), 3.93 (t, 2H), 3.23-3.34 (m, 6H), 2.94 (t, 2H), 2.55 (bt, 2H), 2.49 (bs, 4H), 2.00 (m, 2H), 1.65 (bt, 4H), 1.47 (bm, 2H); MS (APCI), M+H: 380 (100%).

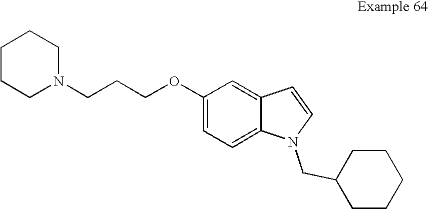

1-Cyclohexylmethyl-5-(3-piperidin-1-yl-propoxy)-1H-indole; prepared from 5-(3-piperidin-1-yl-propoxy)-2,3-dihydro-1H-indole dihydrochloride (30 mg, 0.09 mmol) and cyclohexanecarboxaldehyde(22 μL, 0.18 mmol) by the method of Procedure J (See herein Example 23). The crude material was passed through a short plug of silica gel (20:1 DCM/7N NH₃ in methanol) and further purified by preparative TLC (20:1 DCM/7N NH₃ in methanol) to give the title compound as a brown oil (9 mg, 28%). ¹H NMR (CDCl₃) δ 7.20 (d, 1H), 7.08 (d, 1H), 7.02 (d, 1H), 6.85 (dd, 1H), 6.37 (d, 1H), 4.04 (t, 2H), 3.89 (d, 2H), 3.86 (t, 2H), 2.54 (t, 2H), 2.45 (bm, 4H), 2.02 (m, 2H), 1.82 (m, 1H), 1.57-1.70 (m, 8H), 1.45 (m, 2H), 1.16 (m, 4H), 0.97 (m, 2H); MS (APCI), M+H: 355 (100%).

TABLE 1

| Example | |
|---|---|
| Number | Structure |
| 1 | 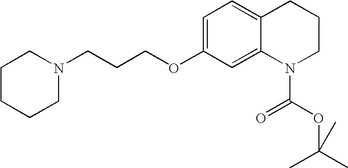 |
| 2 | 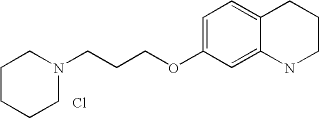 |
| 3 | 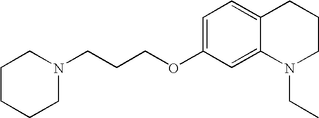 |
| 4 | 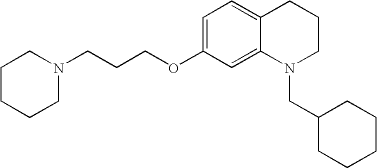 |
| 5 | 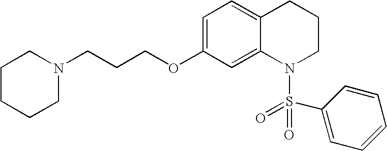 |
| 6 | 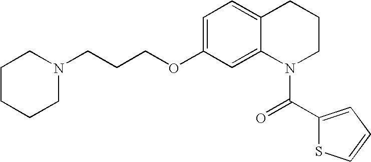 |
| 7 | 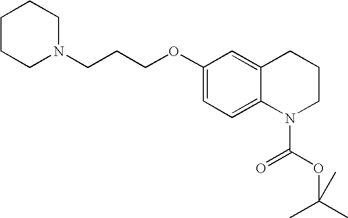 |
| 8 | 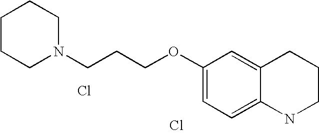 |
| 9 | 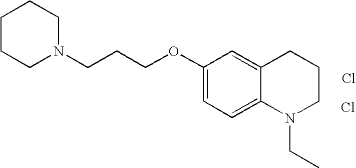 |
| 10 | 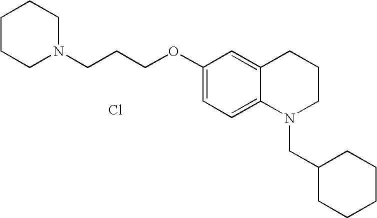 |
| 11 | 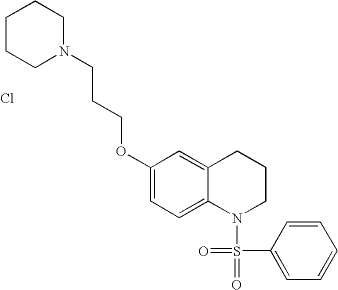 |
| 12 | 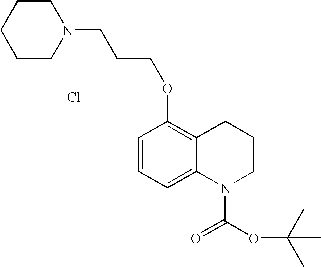 |
| 13 | 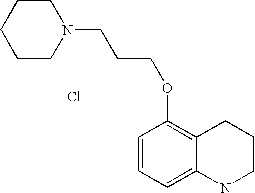 |
| 14 | 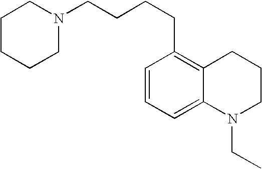 |
| 15 | 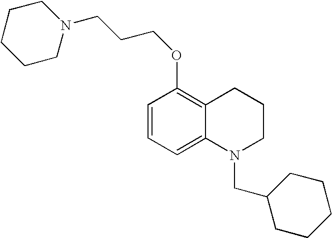 |
| 16 | 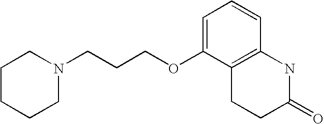 |
| 17 | 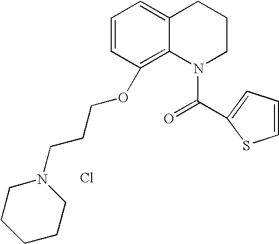 |
| 18 | 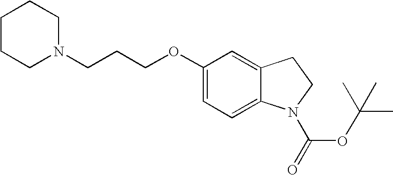 |
| 19 | 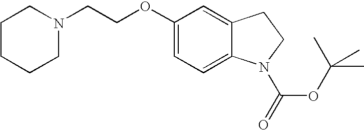 |
| 20 | 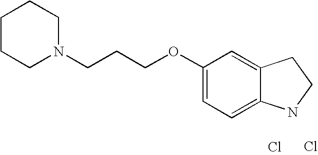 |
| 21 | 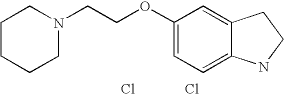 |
| 22 | 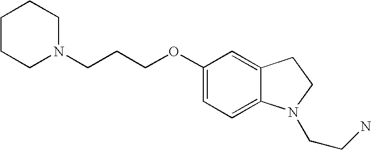 |
| 23 | 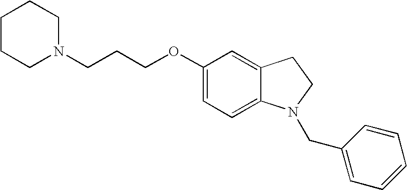 |
| 24 | 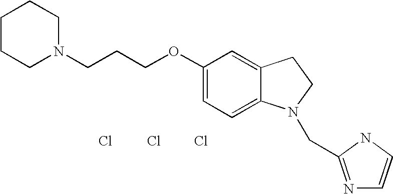 |
| 25 | 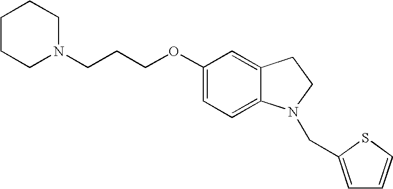 |
| 26 | 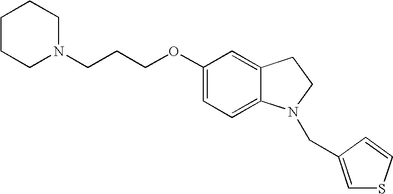 |
| 27 | 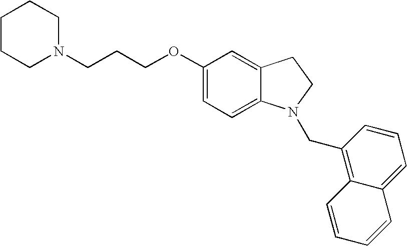 |
| 28 | 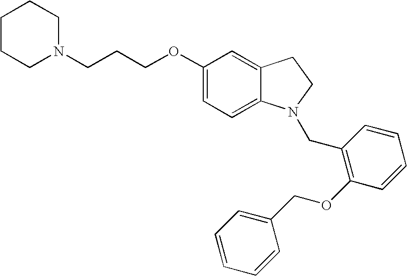 |
| 29 | 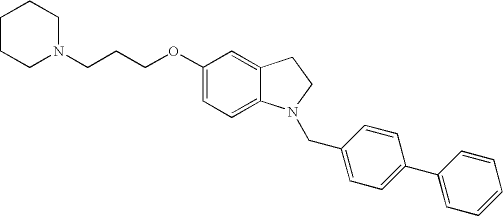 |
| 30 | 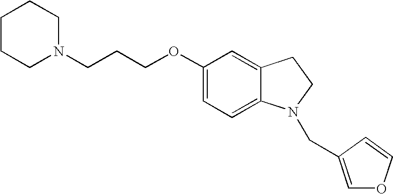 |
| 31 | 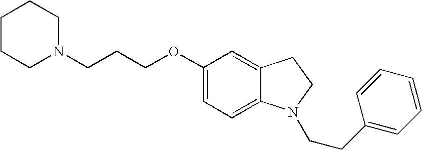 |
| 32 | 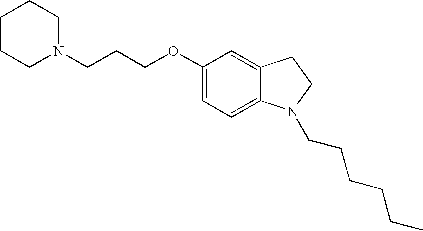 |
| 33 | 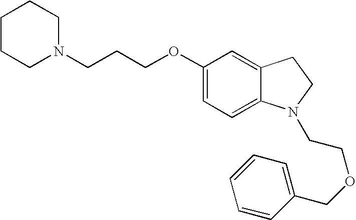 |
| 34 | 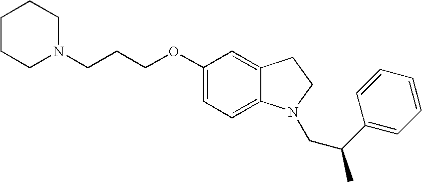 |
| 35 | 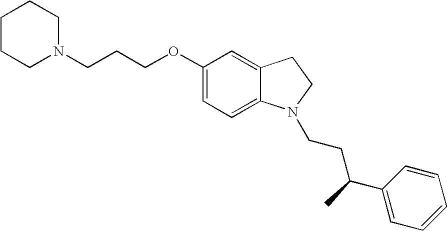 |
| 36 | 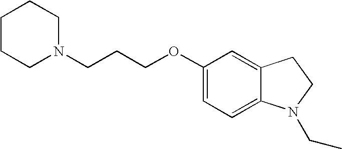 |
| 37 | 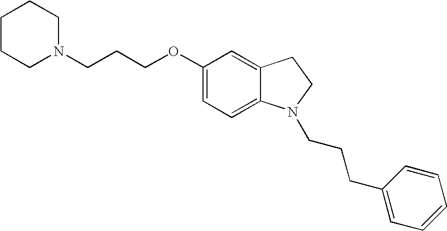 |
| 38 | 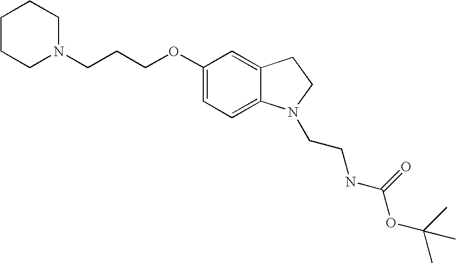 |
| 39 | 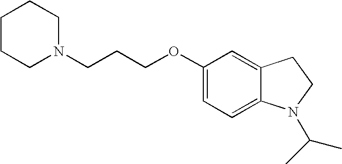 |
| 40 | 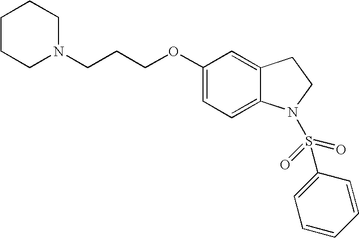 |
| 41 | 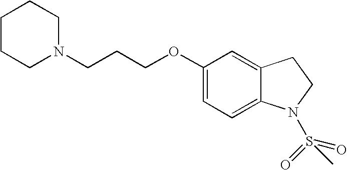 |
| 42 | 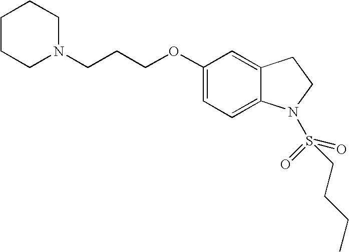 |
| 43 | 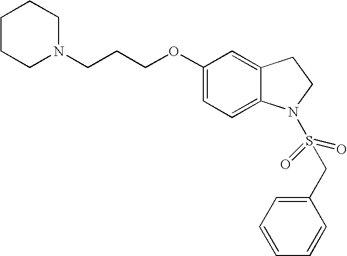 |
| 44 | 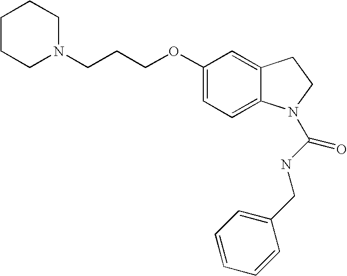 |
| 45 | 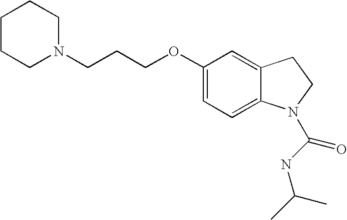 |
| 46 | 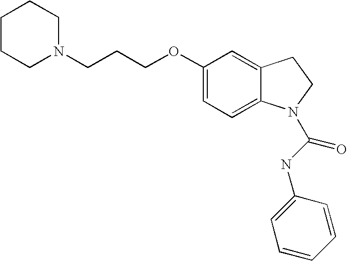 |
| 47 | 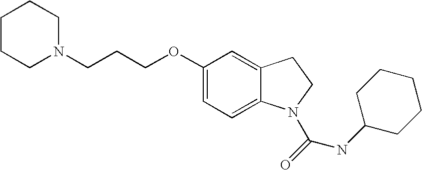 |
| 48 | 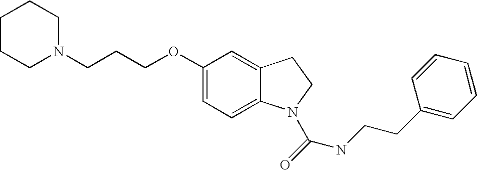 |
| 49 | 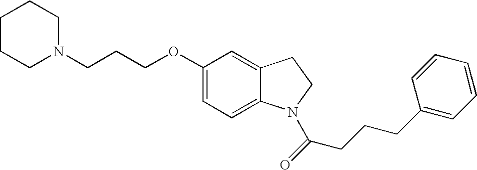 |
| 50 | 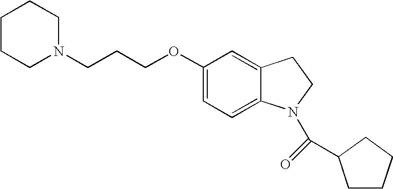 |
| 51 | 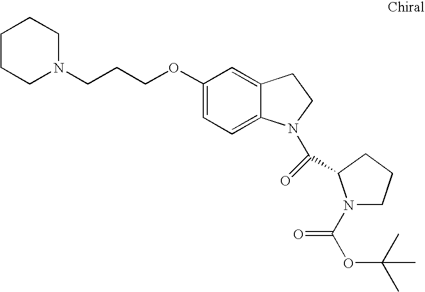 |
| 52 | 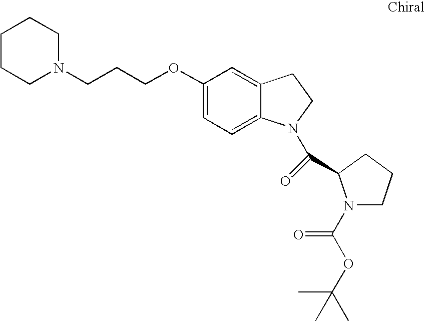 |
| 53 | 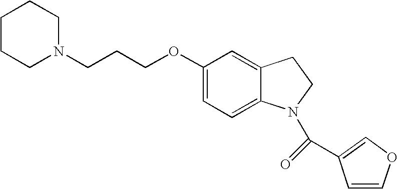 |
| 54 | 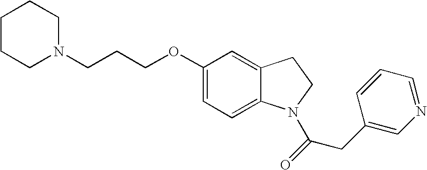 |
| 55 | 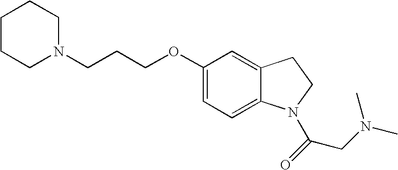 |
| 56 | 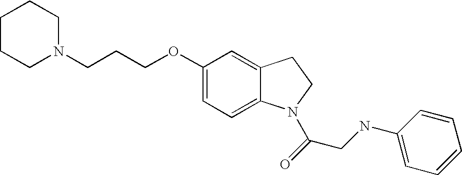 |
| 57 | 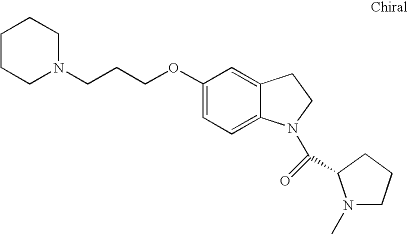 |
| 58 | 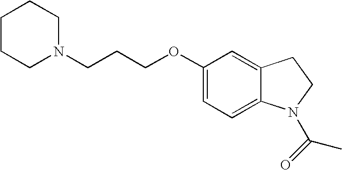 |
| 59 | 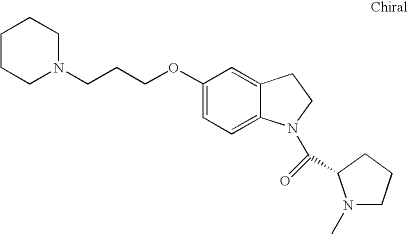 |
| 60 | 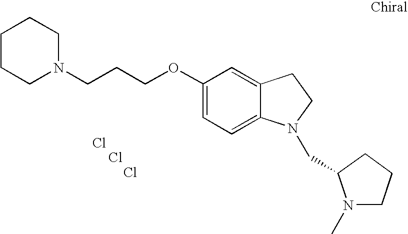 |
| 61 | 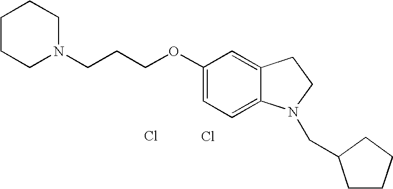 |
| 62 | 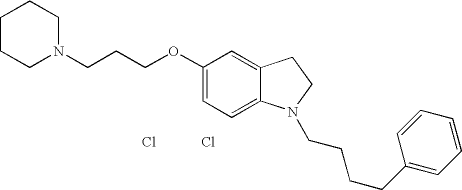 |
| 63 | 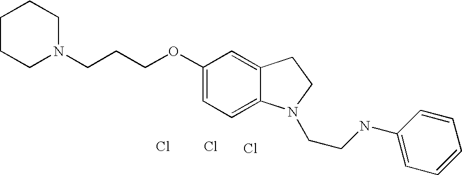 |
| 64 | 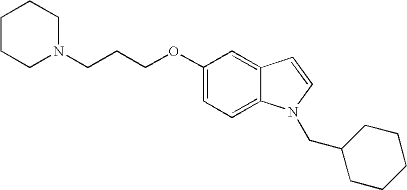 |

The pharmaceutical salts of the invention are typically formed by reacting a compound of Formula I with an equimolar or excess amount of acid or base. The reactants are generally combined in a mutual solvent such as diethylether, tetrahydrofuran, methanol, ethanol, isopropanol, benzene, and the like for acid addition salts, or water, an alcohol or a chlorinated solvent such as dichloromethane for base addition salts. The salts normally precipitate out of solution within about one hour to about ten days and can be isolated by filtration or other conventional methods.

Acids commonly employed to form pharmaceutical acid addition salts are inorganic acids such as hydrochloric acid, hydrobromic acid, hydroiodic acid, sulfuric acid, phosphoric acid, and the like, and organic acids such as p-toluenesulfonic, methanesulfonic acid, ethanesulfonic acid, oxalic acid, p-bromophenylsulfonic acid, carbonic acid, succinic acid, citric acid, tartaric acid, benzoic acid, acetic acid, and the like. Preferred pharmaceutical acid addition salts are those formed with mineral acids such as hydrochloric acid, hydrobromic acid, and sulfuric acid, and those formed with organic acids such as maleic acid, tartaric acid, and methanesulfonic acid.

Bases commonly employed to form pharmaceutical base addition salts are inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, sodium carbonate, sodium bicarbonate, potassium bicarbonate, calcium hydroxide, calcium carbonate, and the like. The potassium and sodium salt forms are particularly preferred.

It should be recognized that the particular counterion forming a part of any salt of this invention is not of a critical nature, so long as the salt as a whole is pharmacologically acceptable and as long as the counterion does not contribute undesired qualities to the salt as a whole.

The optimal time for performing the reactions of the Schemes and the Route can be determined by monitoring the progress of the reaction via conventional chromatographic techniques. Furthermore, it is preferred to conduct the reactions of the invention under an inert atmosphere, such as, for example, argon, or, particularly, nitrogen. Choice of solvent is generally not critical so long as the solvent employed is inert to the ongoing reaction and sufficiently solubilizes the reactants to effect the desired reaction. The compounds are preferably isolated and purified before their use in subsequent reactions. Some compounds may crystallize out of the reaction solution during their formation and then collected by filtration, or the reaction solvent may be removed by extraction, evaporation, or decantation. The intermediates and final products of Formula I may be further purified, if desired by common techniques such as recrystallization or chromatography over solid supports such as silica gel or alumina.

The skilled artisan will appreciate that not all substituents are compatible with all reaction conditions. These compounds may be protected or modified at a convenient point in the synthesis by methods well known in the art.

The compound of Formula I is preferably formulated in a unit dosage form prior to administration. Therefore, yet another embodiment of the present invention is a pharmaceutical composition comprising a compound of Formula I and one or more pharmaceutically acceptable carriers, diluents or excipients.

The present pharmaceutical compositions are prepared by known procedures using well-known and readily available ingredients. In making the formulations of the present invention, the active ingredient (Formula I compound) will usually be mixed with a carrier, or diluted by a carrier, or enclosed within a carrier which may be in the form of a capsule, sachet, paper or other container. When the carrier serves as a diluent, it may be a solid, semisolid or liquid material that acts as a vehicle, excipient, or medium for the active ingredient. Thus, the compositions can be in the form of tablets, pills, powders, lozenges, sachets, cachets, elixirs, suspensions, emulsions, solutions, syrups, aerosol (as a solid or in a liquid medium), soft and hard gelatin capsules, suppositories, sterile injectable solutions and sterile packaged powders.

Some examples of suitable carriers, excipients, and diluents include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water syrup, methyl cellulose, methyl and propylhydroxybenzoates, talc, magnesium stearate and mineral oil. The formulations can additionally include lubricating agents, wetting agents, emulsifying and suspending agents, preserving agents, sweetening agents or flavoring agents. The compositions of the invention may be formulated so as to provide quick, sustained or delayed release of the active ingredient after administration to the patient.

The compositions of the present invention may be formulated in sustained release form to provide the rate controlled release of any one or more of the components or active ingredients to optimize the therapeutic effects, i.e., antihistaminic activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

Liquid form preparations include solutions, suspensions and emulsions. As an example may be mentioned water or water-propylene glycol solutions for parenteral injections or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier such as inert compressed gas, e.g. nitrogen.

For preparing suppositories, a low melting wax such as a mixture of fatty acid glycerides such as cocoa butter is first melted, and the active ingredient is dispersed homogeneously therein by stirring or similar mixing. The molten homogeneous mixture is then poured into convenient sized molds, allowed to cool and thereby solidify.

Also included are solid form preparations which are intended to be converted, shortly before use, to liquid form preparations for either oral or parenteral administration, Such liquid forms include solutions, suspensions and emulsions.

The compounds of the invention may also be deliverable transdermally. The transdermal compositions may take the form of creams, lotions, aerosols and/or emulsions and can be included in a transdermal patch of the matrix or reservoir type as a re conventional in the art for this purpose.

Preferably the compound is administered orally.

Preferably, the pharmaceutical preparation is in a unit dosage form. In such form, the preparation is subdivided into suitably sized unit doses containing appropriate quantities of the active components, e.g., an effective amount to achieve the desired purpose.

The quantity of the inventive active composition in a unit dose of preparation may be generally varied or adjusted from about 0.01 milligrams to about 1,000 milligrams, preferably from about 0.01 to about 950 milligrams, more preferably from about 0.01 to about 500 milligrams, and typically from about 1 to about 250 milligrams, according to the particular application. The actual dosage employed may be varied depending upon the patient's age, sex, weight and severity of the condition being treated. Such techniques are well known to those skilled in the art. Generally, the human oral dosage form containing the active ingredients can be administered 1 or 2 times per day.

Utility

Compounds of Formula I are effective as histamine H3 receptor antagonists. More particularly, these compounds are selective histamine H3 receptor antagonists that have little or no affinity for histamine receptor GPRv53 (H4R). As selective antagonists, the compounds of Formula I are useful in the treatment of diseases, disorders, or conditions responsive to the inactivation of the histamine H3 receptor, including but not limited to obesity and other eating-related disorders. It is postulated that selective antagonists of H3R will raise brain histamine levels and possibly that of other monoamines resulting in inhibition of food consumption while minimizing peripheral consequences. Although a number of H3R antagonists are known in the art, none have proven to be satisfactory obesity drugs. There is increasing evidence that histamine plays an important role in energy homeostasis. Histamine, acting as a neurotransmitter in the hypothalamus, suppressed appetite. Histamine is an almost ubiquitous amine found in many cell types and it binds to a family of G protein-coupled receptors (GPCRs). This family provides a mechanism by which histamine can elicit distinct cellular responses based on receptor distribution. Both the H1R and H2R are widely distributed. H3R is primarily expressed in the brain, notably in the thalamus and caudate nucleus. High density of expression of H3R was found in feeding center of the brain. A novel histamine receptor GPRv53 has been recently identified. GPRv53 is found in high levels in peripheral white blood cells; only low levels have been identified in the brain by some investigators while others cannot detect it in the brain. However, any drug discovery effort initiated around H3R must consider GPRv53 as well as the other subtypes.

The inventive compounds can readily be evaluated by using a competitive inhibition Scintillation Proximity Assay (SPA) based on a H3R binding assay using [3H] (x methylhistamine as ligand. Stable cell lines, including but not limited to HEK can be transfected with cDNA coding for H3R to prepare membranes used for the binding assay. The technique is illustrated below (Preparation of Histamine Receptor Subtype Membranes) for the histamine receptor subtypes.

Membranes isolated as described in (Preparation of Histamine Receptor Subtype Membranes) were used in a [³⁵S]GTPχS functional assay. Binding of [³⁵S]GTPχS to membranes indicates agonist activity. Compounds of the invention of Formula I were tested for their ability to inhibit binding in the presence of agonists. Alternately, the same transfected cell lines were used for a cAMP assay wherein H3R agonists inhibited forskolin-activated synthesis of cAMP. Compounds of Formula I were tested for their ability to permit forskolin-stimulated cAMP synthesis in the presence of agonist.

Preparation of Histamine Receptor Subtype Membranes

A. Preparation H1R Membranes

cDNA for the human histamine 1 receptor (H1R) was cloned into a mammalian expression vector containing the CMV promoter (pcDNA3.1(+), Invitogen) and transfected into HEK293 cells using the FuGENE Tranfection Reagent (Roche. Diagnostics Corporation). Transfected cells were selected using G418 (500 μ/ml). Colonies that survived selection were grown and tested for histamine binding to cells grown in 96-well dishes using a scintillation proximity assay (SPA) based radioligand binding assay. Briefly, cells, representing individual selected clones, were grown as confluent monolayers in 96-well dishes (Costar Clear Bottom Plates, #3632) by seeding wells with 25,000 cells and growing for 48 hours (37° C., 5% CO₂). Growth media was removed and wells were rinsed two times with PBS (minus Ca²⁺ or Mg²⁺). For total binding, cells were assayed in a SPA reaction containing 5 mM Tris-HCL (assay buffer), pH 7.6, 1 mg wheat germ agglutinin SPA beads (Amersham Pharmacia Biotech, #RPNQ0001), and 0.8 nM ³H-pyrilamine (Net-594, NEN) (total volume per well=200 μl). Astemizole (10 μM, Sigma #A6424) was added to appropriate wells to determine non-specific binding. Plates were covered with FasCal and incubated at room temperature for 120 minutes. Following incubation, plates were centrifuged at 1,000 rpm (˜800 g) for 10 minutes at room temperature. Plates were counted in a Wallac Trilux 1450 Microbeta scintillation counter. Several clones were selected as positive for binding, and a single clone (H1R40) was used to prepare membranes for binding studies. Cell pellets, representing ˜10 grams, were resuspended in 30 ml assay buffer, mixed by vortexing, and centrifuged (40,000 g at 4° C.) for 10 minutes. The pellet resuspension, vortexing, and centrifugation was repeated 2 more times. The final cell pellet was resuspended in 30 ml and homogenized with a Polytron Tissue Homogenizer. Protein determinations were done using the Coomassie Plus Protein Assay Reagent (Pierce). Five micrograms of protein was used per well in the SPA receptor-binding assay.

B. Preparation H2R Membranes

cDNA for the human histamine 2 receptor was cloned, expressed and transfected into HEK 293 cells as described above. Histamine binding to cells was assayed by SPA described above. For total binding, cells were assayed in a SPA reaction containing 50 mM Tris-HCl (assay buffer), pH 7.6, 1 mg wheat germ agglutinin SPA beads (Amersham Pharmacia Biotech, #RPNQ0001), and 6.2 nM ³H-tiotidine (Net-688, NEN) (total volume per well=200 μl). Cimetidine (10 μM, Sigma #C4522) was added to appropriate wells to determine non-specific binding.

Several clones were selected as positive for binding, and a single clone (H2R10) was used to prepare membranes for binding studies. Five micrograms of protein was used per well in the SPA receptor-binding assay.

C. Preparation of H3R Membranes

cDNA for the human histamine 3 receptor was cloned and expressed as described in (A. Preparation H1R membranes), above. Transfected cells were selected using G418 (500 μ/ml), grown, and tested for histamine binding by the SPA described above. For total binding, cells were assayed in a SPA reaction described above containing 50 mM Tris-HCL (assay buffer), pH 7.6, 1 mg wheat germ agglutinin SPA beads (Amersham Pharmacia Biotech, #RPNQ0001), and 1 nM (³H)-n-alpha-methylhistamine (NEN, NET1027) (total volume per well=200 μl). Thioperimide was added to determine non-specific binding. Several clones were selected as positive for binding, and a single clone (H3R8) was used to prepare membranes for binding studies described above. Five micrograms of protein was used per well in the SPA receptor-binding assay.

All compounds set forth in examples above exhibited affinity for the H3 receptor greater than 1 μM. Preferred compounds of the invention exhibited affinity for the H3 receptor greater than 200 nM. Most preferred compounds of the invention exhibit affinity for the H3 receptor greater than 20 nM.

D. Preparation of GPRv53 Membranes

cDNA for the human GPRv53 receptor was cloned and expressed as described in (A. Preparation H1R membranes), above. Transfected cells were selected, tested for histamine binding, and selected. HEK293 GPRv53 50 cells were grown to confluency in DMEM/F12 (Gibco) supplemented with 5% FBS and 500 ug/ml G418 and washed with Delbecco's PBS (Gibco) and harvested by scraping. Whole cells were homogenized with a Polytron tissuemizer in binding buffer, 50 mM Tris pH 7.5. Cell lysates, 50 ug, were incubated in 96 well dishes with 3 nM (3H) Histamine and compounds in binding buffer for 2 hours at room temperature. Lysates were filtered through glass fiber filters (Perkin Elmer) with a Tomtec cell harvester. Filters were counted with melt-on scintillator sheets (Perkin Elmer) in a Wallac Trilux 1450 Microbeta Scintillation counter for 5 minutes.

Pharmacological Results

cAMP ELISA

HEK293H3R8 cells prepared as described above were seeded at a density of 50,000 cells/well and grown overnight in DMEM/F12 (Gibco) supplemented with 5% FBS and 500 ug/ml G418. The next day tissue culture medium was removed and replaced with 50 μl cell culture medium containing 4 mM 3-isobutyl-1-methylxanthine (Sigma) and incubated for 20 minutes at room temperature. Antagonist were added in 50 μl cell culture medium and incubated for 20 minutes at room temperature. Agonist R (−)α methylhistamine (RBI) at a dose response from 1×10⁻¹⁰ to 1×10⁻⁵ M was then added to the wells in 50 μl cell culture medium and incubated for 5 minutes at room temperature. Then 50 μl of cell culture medium containing 20 μM Forskolin (Sigma) was added to each well and incubated for 20 minutes at room temperature. Tissue culture medium was removed and cells were lysed in 0.1M HCl and cAMP was measured by ELISA (Assay Designs, Inc.).

[³⁵S] GTP γ [S] Binding Assay

Antagonist activity of selected compounds was tested for inhibition of [³⁵S] GTP γ [S] binding to H3R membranes in the presence of agonists. Assays were run at room temperature in 20 mM HEPES, 100 mM NaCl, 5 mM MgCl₂ and 10 uM GDP at pH 7.4 in a final volume of 200 ul in 96-well Costar plates. Membranes isolated from H3R8-expressing HEK293 cell line (20 ug/well) and GDP were added to each well in a volume of 50 μl assay buffer. Antagonist was then added to the wells in a volume of 50 μl assay buffer and incubated for 15 minutes at room temperature. Agonist R(−)alpha methylhistamine (RBI) at either a dose response from 1×10⁻¹⁰ to 1×10⁻⁵ M or fixed concentration of 100 nM were then added to the wells in a volume of 50 μl assay buffer and incubated for 5 minutes at room temperature. GTP γ [³⁵S] was added to each well in a volume of 50 μl assay buffer at a final concentration of 200 pM, followed by the addition of 50 μl of 20 mg/ml WGA coated SPA beads (Amersham). Plates were counted in Wallac Trilux 1450 Microbeta scintillation counter for 1 minute. Compounds that inhibited more than 50% of the specific binding of radioactive ligand to the receptor were serially diluted to determine a K[i](nM). The results are given below for the indicated compound.

TABLE 2

| Example | Ki (nM) |
|---|---|
| 6 | 12.6 |
| 60 | 0.5 |

From the above description, one skilled in the art can ascertain the essential characteristics of the present invention, and without departing from the spirit and scope thereof, can make various changes and modifications of the invention to adapt it to various usages and conditions. Thus, other embodiments are also within the claims.

1. A compound structurally represented by Formula I 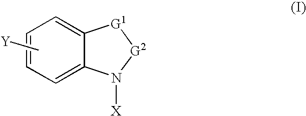
or pharmaceutically acceptable salts thereof wherein:
G¹ is —CH₂—, or —CH₂—CH₂—,
G² is —CH₂—, or —C(O)—,
or G¹ and G² taken together combine to form —CH═CH— or —CH₂—CH═CH—,
Y is
—OCH₂CH₂N-piperidinyl,
—OCH₂CH₂CH₂N-piperidinyl,
—OCH₂CH₂N-pyrrolidinyl,
—OCH₂CH₂CH₂N-pyrrolidinyl,
X is H, —COR³, —CH₂R⁴, —SO₂R⁵,
R³ is
—(C₁-C₈) alkyl, optionally substituted with 1 to 3 halogens,
—(C₃-C₈) cycloalkyl, optionally substituted with 1 to 3 halogens,
—O(C₁-C₈) alkyl, optionally substituted with 1 to 3 halogens, 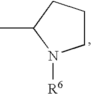
wherein R⁶ is —(C₁-C₆) alkyl, or —COO—(C₁-C₆) alkyl,
Furanyl,
Thienyl,
—NH-phenyl,
—NH—(C₁-C₄)alkyl-phenyl,
—NH—(C₁-C₈) alkyl, optionally substituted with 1 to 4 halogens,
—NH—(C₃-C₈) cycloalkyl, optionally substituted once or twice with halogens,
—CH₂-Pyridinyl, —CH₂N(C₁-C₆) alkyl (C₁-C₆) alkyl,
—CH₂N-phenyl,
R⁴ is
—(C₁-C₈) alkyl, optionally substituted with 1 to 4 halogens,
—(C₃-C₈) cycloalkyl,
—(C₁-C₈) alkyl-NH₂,
—(C₁-C₄) alkyl-phenyl,
—CH₂N-phenyl,
-phenyl-O—(C₁-C₄) alkyl-phenyl,
—(C₁-C₄) alkyl-O—(C₁-C₄) alkyl-phenyl,
—CH₂NCO₂—(C₁-C₆) alkyl,
-Phenyl,
-Thienyl,
-Furanyl,
-Imidazolyl,
-Naphthyl, 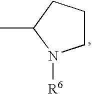
wherein R⁶ is —(C₁-C₆) alkyl, or —COO—(C₁-C₆) alkyl,
-Biphenyl, and
R⁵ is
-Phenyl,
—(C₁-C₄) alkyl,
—(C₁-C₄) alkyl-phenyl.

2. The compound of claim 1, wherein R¹ and R² cyclize to form a 5-membered ring.

3. The compound of claim 1, wherein R¹ and R² cyclize to form a 6-membered ring.

4. The compound of claim 2 wherein Y is in the 5 position.

5. The compound of claim 3 wherein Y is in the 6 position.

6. The compound of claim 4 wherein X is CO.

7. The compound of claim 1, selected from the group consisting of:

| Example | |
|---|---|
| Number | Structure |
| 1 | 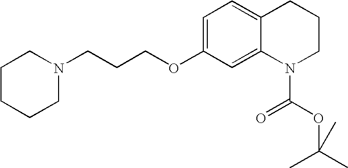 |
| 2 | 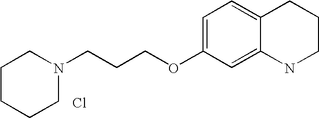 |
| 3 | 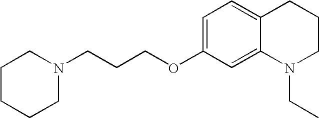 |
| 4 | 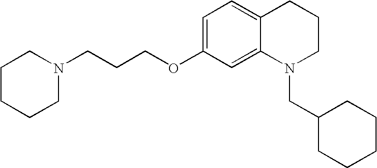 |
| 5 | 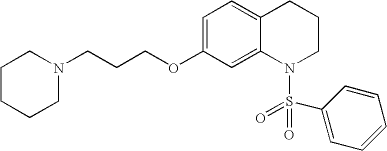 |
| 6 | 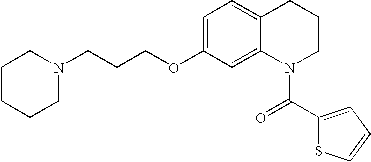 |
| 7 | 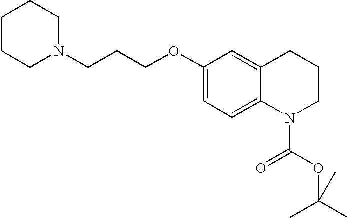 |
| 8 | 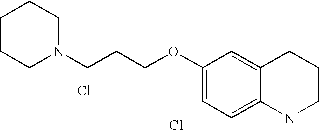 |
| 9 | 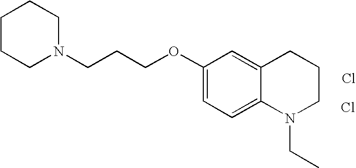 |
| 10 | 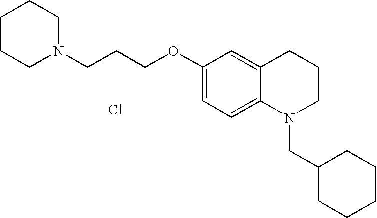 |
| 11 | 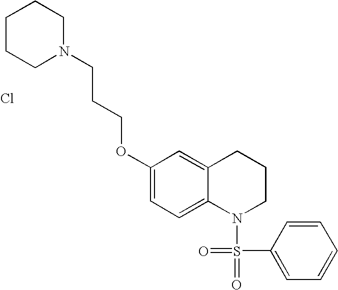 |
| 12 | 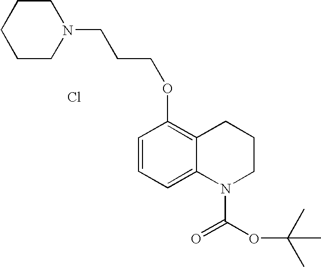 |
| 13 | 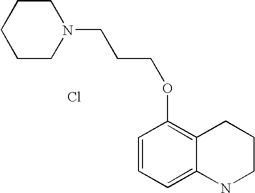 |
| 14 | 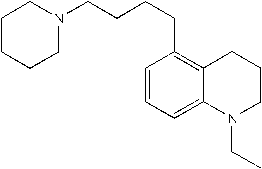 |
| 15 | 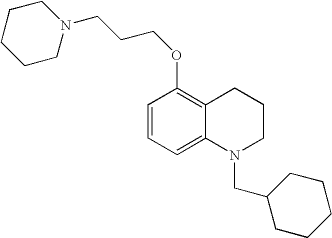 |
| 16 | 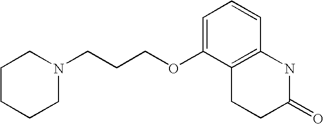 |
| 17 | 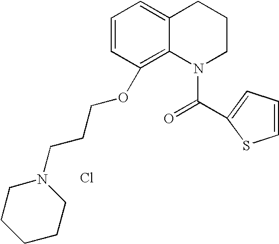 |
| 18 | 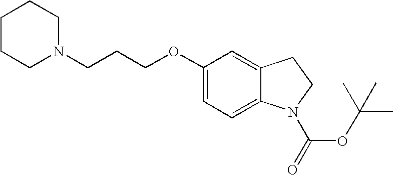 |
| 19 | 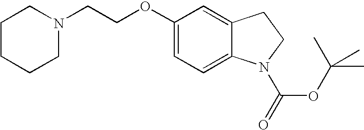 |
| 20 | 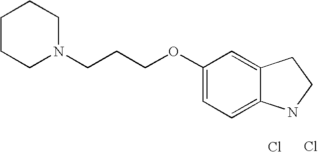 |
| 21 | 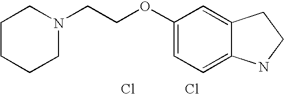 |
| 22 | 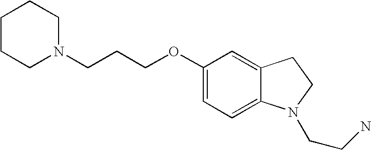 |
| 23 | 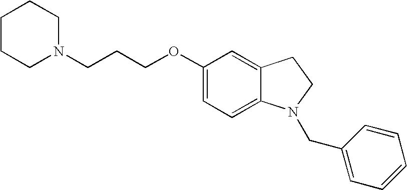 |
| 24 | 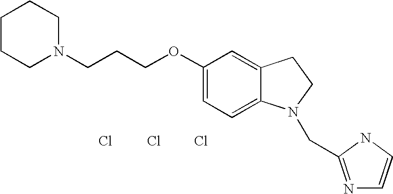 |
| 25 | 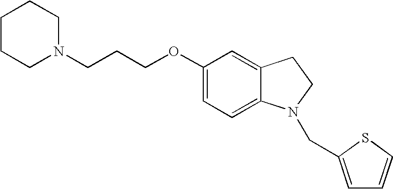 |
| 26 | 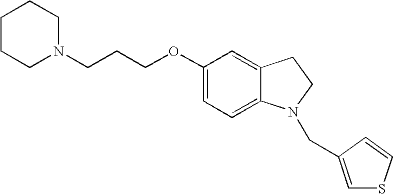 |
| 27 | 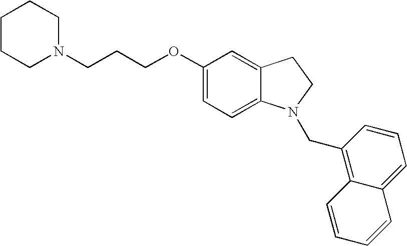 |
| 28 | 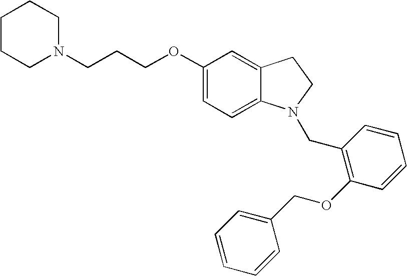 |
| 29 | 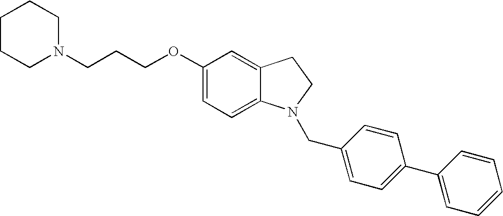 |
| 30 | 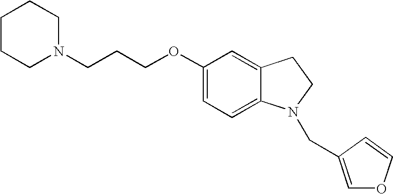 |
| 31 | 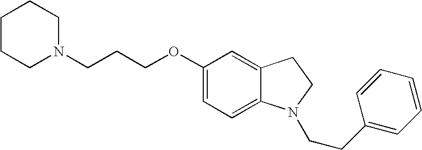 |
| 32 | 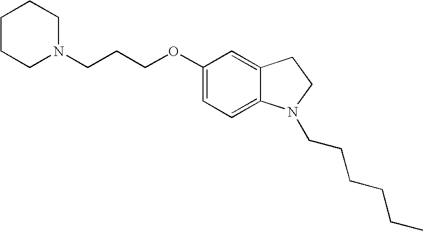 |
| 33 | 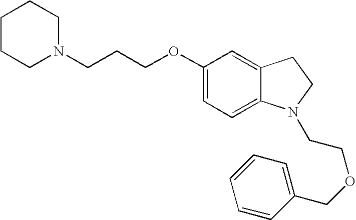 |
| 34 | 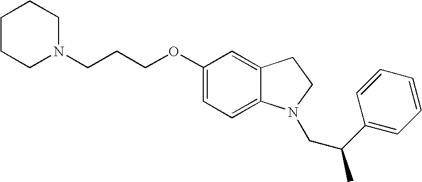 |
| 35 | 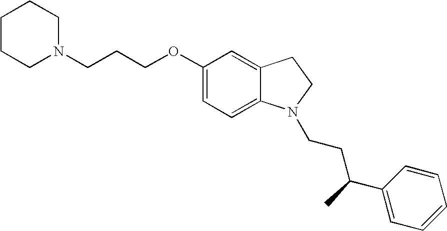 |
| 36 | 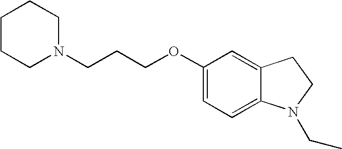 |
| 37 | 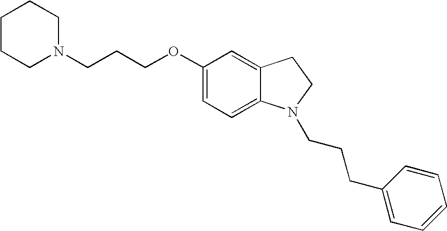 |
| 38 | 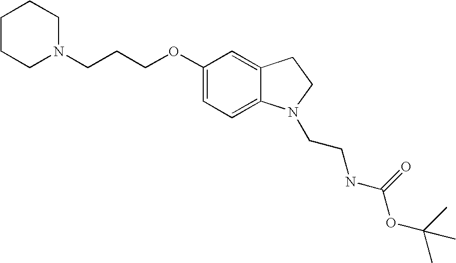 |
| 39 | 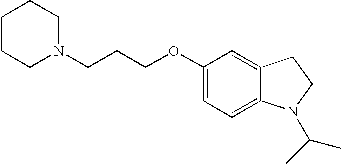 |
| 40 | 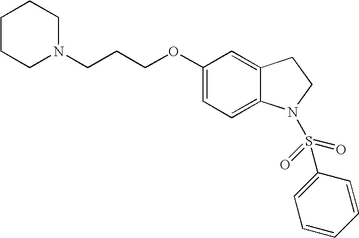 |
| 41 | 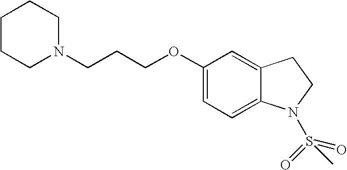 |
| 42 | 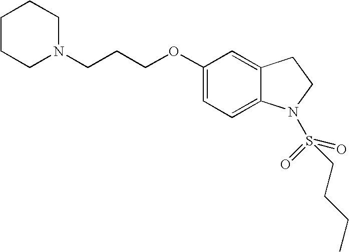 |
| 43 | 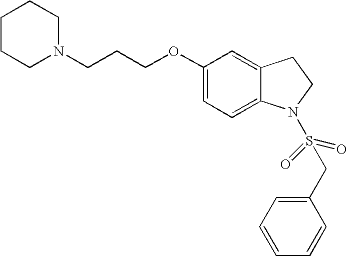 |
| 44 | 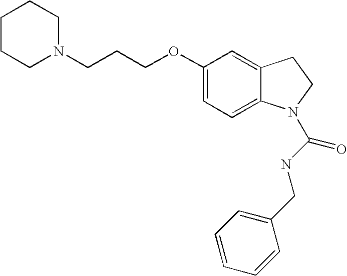 |
| 45 | 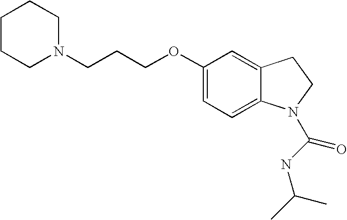 |
| 46 | 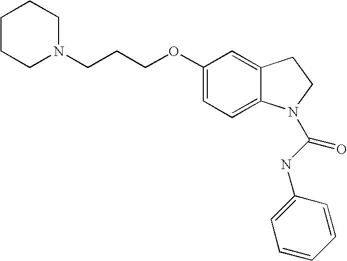 |
| 47 | 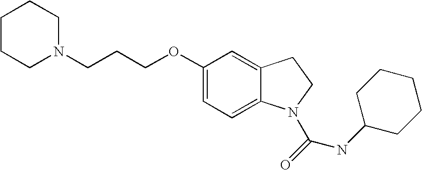 |
| 48 | 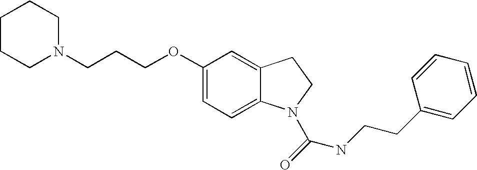 |
| 49 | 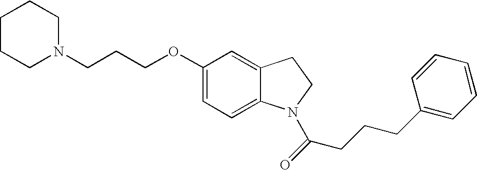 |
| 50 | 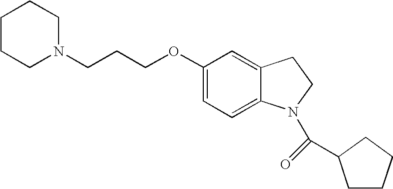 |
| 51 | 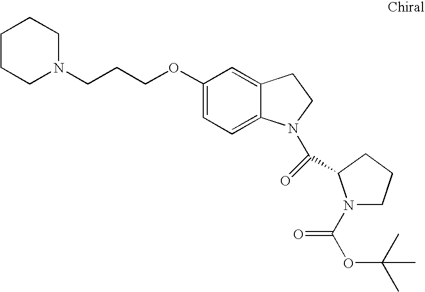 |
| 52 | 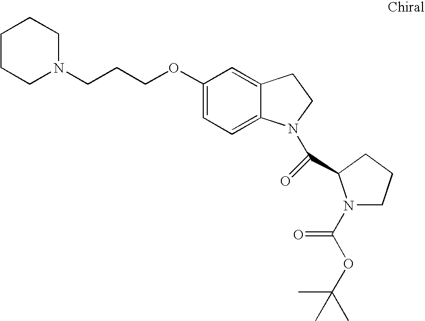 |
| 53 | 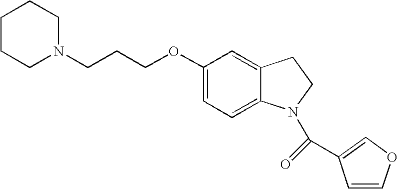 |
| 54 | 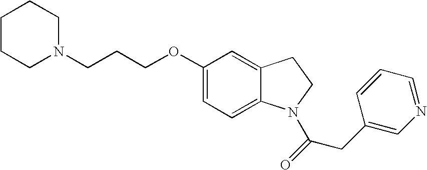 |
| 55 | 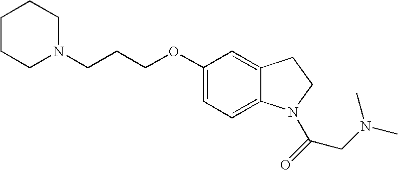 |
| 56 | 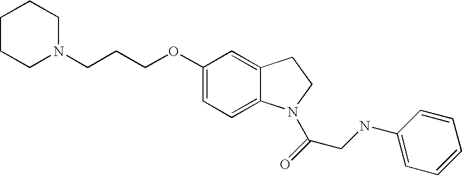 |
| 57 | 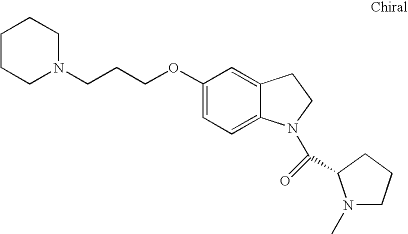 |
| 58 | 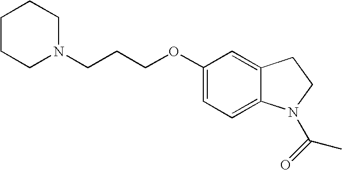 |
| 59 | 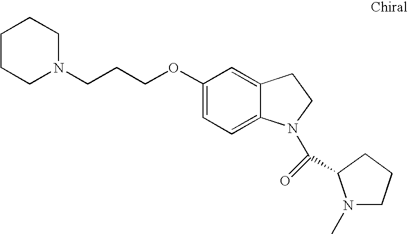 |
| 60 | 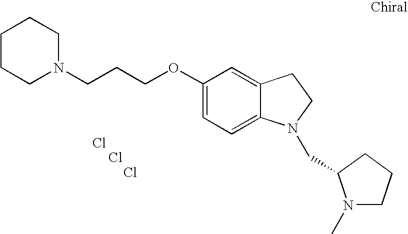 |
| 61 | 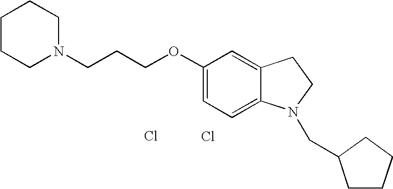 |
| 62 | 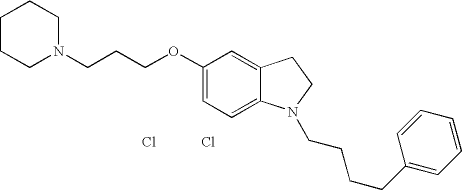 |
| 63 | 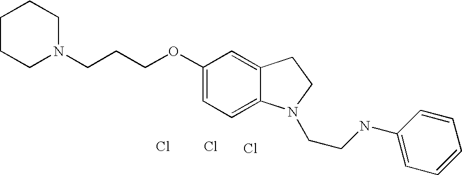 |
| 64 | 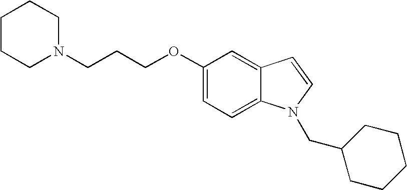 |

or a pharmaceutically acceptable salt or solvate thereof.

8. A pharmaceutical composition which comprises a compound of claims 1 or 7 and a pharmaceutically acceptable carrier.

9. (canceled)

10. (canceled)

11. (canceled)

12. A method for treatment or prevention of obesity which comprises administering to a subject in need of such treatment or prevention an effective amount of a compound of claims 1 or 7.

13. The method of claim 12 wherein the antagonist is a pharmaceutical composition of claim 8.

14. A method for treatment or prevention of a cognitive disorder which comprises administering to a subject in need of such treatment or prevention an effective amount of a compound of claims 1 or 7.

15. The method of claim 15 wherein the antagonist is a pharmaceutical composition of claim 8.
